(19) United States
(12) Patent Application Publication
(10) Pub. No.: US 2024/0041878 A1
(43) Pub. Date: Feb. 8, 2024

(54) APELIN RECEPTOR MODULATORS FOR TREATMENT OF A DISORDER OR DISEASE ASSOCIATED WITH BBB PERMEABILITY

(71) Applicant: BioAge Labs, Inc., Richmond, CA (US)

(72) Inventors: Justin REBO, Derry, NH (US); Yan WANG, Fremont, CA (US); Adam LUNDQUIST, Oakland, CA (US); Matthew BANICKI, Emeryville, CA (US); Robert HUGHES, Berkeley, CA (US); Peng Khun LEONG, San Francisco, CA (US)

(21) Appl. No.: 18/326,934

(22) Filed: May 31, 2023

Related U.S. Application Data
(60) Provisional application No. 63/347,073, filed on May 31, 2022.
(60) Provisional application No. 63/413,430, filed on Oct. 5, 2022.
(60) Provisional application No. 63/478,330, filed on Jan. 3, 2023.
(60) Provisional application No. 63/478,336, filed on Jan. 3, 2023.

(51) Int. Cl. A61K 31/506; A61P 29/00; A61P 25/28

(52) U.S. Cl. CPC A61K 31/506; A61P 29/00; A61P 25/28

(57) ABSTRACT

This disclosure provides methods for using a particular class of apelin receptor modulators to reduce blood-brain barrier (BBB) permeability in a subject in need thereof, and in particular methods of treatment for a variety of disorders, conditions, and diseases associated with and related to increased BBB permeability. This disclosure also provides methods for using a particular class of apelin receptor modulators to treat a neurodegenerative disease, delirium, and/or dementia in a subject in need thereof. This disclosure also provides methods for using a particular class of apelin receptor modulators to reduce neuroinflammation in a subject in need thereof. In some embodiments, the apelin receptor modulator (e.g., agonist) is BGE-105, or a pharmaceutically acceptable salt thereof.

### 1. CROSS-REFERENCE TO RELATED APPLICATIONS

This application claims priority to and benefit of U.S. Provisional Application Nos.: 63/347,073, filed May 31, 2022; 63/413,430, filed Oct. 5, 2022; 63/478,330, filed Jan. 3, 2023; and 63/478,336, filed Jan. 3, 2023; each of which is herein incorporated in its entirety by reference.

### 2. BACKGROUND

Neurodegenerative diseases occur when nerve cells in the brain or peripheral nervous system lose function over time and ultimately die. The likelihood of developing a neurodegenerative disease rises dramatically with age. Common neurodegenerative diseases include Alzheimer disease (AD), Parkinson disease (PD), Huntington disease (HD), amyotrophic lateral sclerosis (ALS), multiple sclerosis (MS), multiple system atrophy (MSA), prion diseases, delirium, dementia, and post-operative cognitive dysfunction.

The blood-brain barrier (BBB) separates the systemic circulation from the brain, regulating transport of most molecules and protecting the brain microenvironment. Central to the BBB are unique features of cerebral endothelial cells, which are adjoined and sealed by specialized junctions (tight junctions, TJ) and exhibit minimal vesicular transport (transcytosis) preventing the passage of hydrophilic molecules from blood to brain and vice versa.

Many diseases and physiological stressors that affect the CNS also alter the functional integrity of the BBB. Changes in the distinct physiological properties of the BBB are associated with BBB breakdown or disruption associated with normal aging, cognitive impairment, and various neurodegenerative disorders and diseases including dementia. These changes can affect the BBB's function in selectively restricting passage of substances from the blood to the brain. BBB disruption can lead to BBB leakage and vascular cognitive impairment.

AD is a common form of dementia. AD brain pathology starts before the onset of clinical symptoms. One early pathological hallmark of AD associated with cognitive decline is BBB dysfunction characterized by barrier leakage.

Additionally, there is increasing appreciation of the role of astrocytes in disorders including AD, PD, HD, and ALS. Increasing evidence indicates that neuroinflammation plays an important role in ALS pathogenesis. Although certain treatments such as anti-inflammatory or immunosuppressive therapies may help relieve some of the physical or mental symptoms associated with neurodegenerative diseases, there remains a need for slowing their progression or curing the diseases.

As the median age of the population increases, there is an increasing need for drugs that reduce or counteract the age-related deficits that lead to cognitive impairment. Therefore, there remains a need for effective therapeutics that can treat disorders and diseases associated with BBB permeability and/or neuroinflammation. Additionally, there remains a need for effective therapeutics that can treat neurodegenerative diseases.

### 3. SUMMARY

An aspect of the present disclosure includes methods for using a particular class of apelin receptor modulators to reduce blood-brain barrier (BBB) permeability in a subject in need thereof, and in particular methods of treatment for a variety of disorders associated with increased or abnormal BBB permeability. In some embodiments, the apelin receptor modulator is an apelin receptor agonist.

Another aspect of the present disclosure includes a method of reducing blood-brain barrier (BBB) permeability in a subject in need thereof, comprising administering to the subject a therapeutically effective amount of an apelin receptor agonist to reduce BBB permeability.

Another aspect of the present disclosure includes a method of treating a disorder related to increased BBB permeability in a subject, comprising administering to a subject in need thereof a therapeutically effective amount of an apelin receptor agonist.

Another aspect of the present disclosure includes a method of treating blood—brain barrier dysfunction in a subject, comprising administering to a subject in need thereof a therapeutically effective amount of an apelin receptor agonist.

Another aspect of the present disclosure includes a method of treating neurodegeneration or a neurodegenerative disease in a subject, comprising administering to a subject in need thereof a therapeutically effective amount of an apelin receptor agonist.

Another aspect of the present disclosure includes a method of reducing neuro-inflammation in a subject, comprising administering to a subject in need thereof a therapeutically effective amount of an apelin receptor agonist.

Another aspect of the present disclosure includes a method of treating a neurodegenerative disease in a subject, comprising administering to a subject in need thereof a therapeutically effective amount of an apelin receptor agonist.

The present inventors applied bioinformatic and machine learning approaches to analyze human data using survival predictor models and discovered an association of apelin protein levels with age-related cognitive decline. We discovered that higher circulating levels of apelin are significantly associated with reduced cognitive decline according to cognitive abilities screening instrument (CAST) score.

The present inventors tested a modulator of the apelin receptor, BGE-105, for its effect on normal adult or aged mice in models of BBB permeability. BGE-105 has the structure shown below:

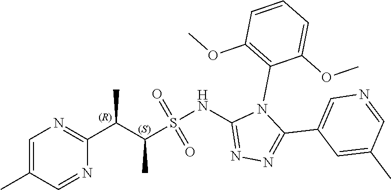

BGE-105 (also referred to as AMG-986) is known to activate the apelin receptor and induces a cardiovascular response in rats (Ason et al., JCI Insight. 5(8):1-16(2020)). Clinical trials were performed with AMG-986 to study the safety, tolerability, and pharmacokinetics in healthy subjects and heart failure subjects (NCT03276728) those with impaired renal function (NCT03318809). Nevertheless, the compound's effect on BBB permeability function in elderly individuals is unknown.

Apelin is a peptide hormone widely expressed throughout the body that signals through its G_{i/o} protein-coupled receptor APJ to exert multiple beneficial effects on cellular function. Within the central nervous system, APJ is primarily expressed in astrocytes, which play important roles in age-related neuroinflammation and neurodegeneration. The present inventors found that in preclinical models of neurodegeneration, direct brain administration of apelin peptide has disease-modifying effects through its effects on apoptosis, inflammation, and autophagy. The present inventors combined multi-omic and computational analysis of proprietary, longitudinal human aging cohorts to identify a novel connection between higher circulating levels of apelin peptide and preservation of cognitive function. The present inventors also observed that apelin pathway activity decreases with age. Based on the connections between apelin and cognitive aging, the relationship between apelin peptide, inflammation, and neurodegeneration, and the expression of APJ on endothelial cells, the present inventors hypothesized that apelin pathway activation could decrease inflammatory signaling in astrocytes and improve blood-brain barrier integrity in aged mice.

In a first set of experiments, the present inventors demonstrated that normal mice (12-month-old) treated with BGE-105 exhibit a significant reversal of LPS-induced BBB permeability, demonstrating in vivo activity in a model of acute cognitive impairment.

In a second set of experiments, BGE-105 reversed BBB permeability in aged mice (26-month-old), demonstrating in vivo activity in a model of age-related cognitive impairment.

In a third set of experiments, BGE-105 improved cellular function in astrocytes and protected against reactive astrocyte cocktail (RAC)-induced cell death in neurons. The data demonstrate BG5-105 is effective in reducing neurotoxicity in degenerative or aging astrocytes, and that BGE-105 could be for treating neurodegenerative diseases and/or related neurodegenerative conditions.

Thus, an exemplary apelin receptor modulator reduced BBB permeability, improves cellular function in astrocytes, thereby protecting neurons against immune activating or inflammatory reactive astrocyte cocktail (RAC)-induced cell death in in vitro and in vivo in models of cognitive impairment, including acute or age-related cognitive impairment. These results indicate that apelin receptor modulators, such as BGE-105, would be effective at reducing BBB permeability and restoring the cellular function of astrocytes to treat acute delirium in a patient, or neurodegenerative disease or dementia in an aged patient.

Accordingly, a first aspect of the present disclosure provides a method of reducing blood-brain barrier (BBB) permeability in a subject in need thereof, the method including administering to the subject a therapeutically effective dose of an apelin receptor modulator. In some aspects of the invention the modulator is an apelin receptor agonist, such as an apelin receptor agonist of formula (I) or (II) as described herein. In some embodiments, the apelin receptor agonist is BGE-105, or a pharmaceutically acceptable salt thereof.

In another aspect, the present disclosure provides a method for treating a disorder associated with increased or abnormal BBB permeability in a subject, the method comprising administering to a subject in need thereof a therapeutically effective dose of an apelin receptor agonist, such as an apelin receptor agonist of formula (I) or (II) as described herein. In some embodiments, the apelin receptor agonist is BGE-105, or a pharmaceutically acceptable salt thereof.

In some embodiments of the methods of this disclosure, the subject is human and has, or is identified as having or exhibiting, cognitive impairment. In some embodiments, the disorder associated with increased or abnormal BBB permeability is acute cognitive impairment, such as postoperative cognitive dysfunction (POCD), or intensive care unit delirium. In some embodiments, the disorder associated with increased or abnormal BBB permeability is a neurodegenerative disease, such as dementia, e.g., Alzheimer's disease, or vascular dementia. In various embodiments, the neurodegenerative disease is selected from Alzheimer's disease (AD), Parkinson's disease (PD), amyotrophic lateral sclerosis (ALS), stroke, Huntington's disease (HD), multiple sclerosis (MS), traumatic brain injury (TBI), dementia, and/or inflammation (e.g., neuroinflammation, peripheral inflammation, etc.).

In another aspect, the present disclosure provides a method of reducing neuro-inflammation in a subject, comprising administering to a subject in need thereof a therapeutically effective amount of an apelin receptor agonist, such as an apelin receptor agonist of formula (I) or (II) as described herein. In some embodiments, the apelin receptor agonist is BGE-105, or a pharmaceutically acceptable salt thereof.

In another aspect, the present disclosure provides a method of reducing and/or treating a neurodegenerative disease in a subject, comprising administering to a subject in need thereof a therapeutically effective amount of an apelin receptor agonist, such as an apelin receptor agonist of formula (I) or (II) as described herein. In some embodiments, the apelin receptor agonist is BGE-105, or a pharmaceutically acceptable salt thereof.

In some embodiments of the methods of this disclosure, the subject is human and has, or is identified as having or exhibiting, cognitive impairment. In some embodiments, the subject has increased neurotoxicity and/or neuroinflammation detected in astrocytic cells, increased astrocyte cytokine or chemokine release, activated or increased NF-κB signaling transcriptional response, and/or glutamate clearance deficiency. In some embodiments, the subject has altered expression (increased or decreased) of one or more immune biomarkers such as cytokines or chemokines (e.g., CCL2, IL-10, CCL11, CCL5, CXCL1, IL-6, CXCL11). In various embodiments, the subject is aged. In various embodiments, the subject has increased BBB permeability (e.g., increased BBB leakage) and/or astrocyte neurotoxicity (e.g., neuroinflammation). In various embodiments, the subject has one or more neurodegenerative diseases selected from Alzheimer's disease (AD), Parkinson's disease (PD), amyotrophic lateral sclerosis (ALS), stroke, Huntington's disease (HD), multiple sclerosis (MS), traumatic brain injury (TBI), dementia, and/or inflammation (e.g., neuroinflammation, peripheral inflammation, etc.).

In another aspect, the present disclosure provides methods for using a particular class of apelin receptor modulators to treat a neurodegenerative disease in a subject in need thereof. In some embodiments, the apelin receptor modulator is an apelin receptor agonist.

In various embodiments, the subject has one or more neurodegenerative diseases selected from Alzheimer's disease (AD), Parkinson's disease (PD), amyotrophic lateral sclerosis (ALS), stroke, Huntington's disease (HD), multiple sclerosis (MS).

In another aspect, the present disclosure provides methods for using a particular class of apelin receptor modulators to treat dementia or delirium in a subject in need thereof. In some embodiments, the apelin receptor modulator is an apelin receptor agonist.

In another aspect, the present disclosure provides methods for using a particular class of apelin receptor modulators to treat traumatic brain injury (TBI) in a subject in need thereof. In some embodiments, the apelin receptor modulator is an apelin receptor agonist.

In another aspect, the present disclosure provides methods for using a particular class of apelin receptor modulators to reduce neuroinflammation in a subject in need thereof. In some embodiments, the apelin receptor modulator is an apelin receptor agonist.

### 4. BRIEF DESCRIPTION OF THE DRAWINGS

These and other features, aspects, and advantages of the present invention will become better understood with regard to the following description, and accompanying drawings, where:

FIG. 1 shows the structure of BGE-105.

FIGS. 2A-2B show a Kaplan Meir curve of the bioinformatic analysis illustrating the relationship between apelin and cognitive decline. The Y-axis represents the probability for CASI decline. The X represents time in years (10 yrs). The purple line represents cohort participants whose APLN level was in the lower 10% of the cohort, while the pink line represents the participants with the highest APLN levels (upper 10%), and the grey line is between 10-90%.

FIGS. 3A-3B show reduced BBB permeability following administration of BGE-105 in an LPS-induced BBB mouse model. The BBB permeability model was established by intraperitoneal injection of 3 mg/kg LPS. FIG. 3A, Mice were divided into three Groups: Group 1: mice administered P.O. vehicle “Control”, Group 2: mice administered P.O. vehicle for 1 week followed by LPS on day 8, and Group 3: mice administered with BGE-105 for 1 week followed by LPS on day 8. Mice in group 3 were administered with BGE-105 at 50 mg/kg (BID) for one week (7 days) before LPS challenge on day 8. The healthy control group mice received P.O. vehicle (BID) for one week (7 days), then followed by one intraperitoneal injection of normal saline. P.O. vehicle or BGE-105 were kept until the endpoint Evans blue assay to measure BBB permeability. 23 hours after LPS or normal saline injection, mice were intravenously injected Evans Blue (EB) (2%, 75 ul/30 g BW). Mice were euthanized before the whole brain was resected to allow for the microdissection of the specific brain regions (olfactory bulb, hippocampus) from each hemisphere. FIG. 3B shows that mice treated with BGE-105 exhibit a significant reversal of the LPS-induced BBB permeability as compared to vehicle treated mice (LPS versus LPS+BGE105) in the olfactory bulb (OB) and hippocampus (HPF).

FIGS. 4A-4C show reduction of BBB permeability following treatment of BGE-105 in aged mice. FIG. 4A shows the difference in blood brain barrier permeability in 3-month old mice (young), 13 month old mice (Adult), and 22-month old mice (Aged). As shown in naturally aged mice, BBB permeability is increased in both the olfactory bulb and hippocampus regions of the brain, as compared to young and adult mice. The activity of BGE-105 on BBB permeability was assessed in aged mice exhibiting age-related increase in BBB permeability according to schematic of FIG. 4B. FIG. 4C shows that pretreatment of BGE-105 significantly reversed the age-induced increase in BBB permeability in the OB as compared to HPF (−28%; p<0.01, Mann-Whitney U-test).

FIG. 5 shows that BGE-105 decreased circulating levels of CXCL1, a peripheral inflammatory marker, in aged mice.

FIG. 6 shows that BGE-105 decreased circulating levels of CXCL13, a peripheral inflammatory marker, in aged mice.

FIG. 7 shows that BGE-105 increased the concentration of total Brain-derived neurotrophic factor (BDNF) expression in the hippocampus in aged mice.

FIG. 8 shows that BGE-105 attenuated RAC-induced CXCL1, CD3 and IL-6 gene expression. CXCL1 expression was significantly upregulated in RAC-induced reactive astrocytes (RAC) by about 1200-1600 fold (p<0.0001) when compared to the control (CON) (left panel). Treatment with BGE-105 (RAC+105) significantly reduced biomarker CXCL1 expression when compared to the RAC group (p=0.0204). C3 expression was significantly upregulated in RAC-induced reactive astrocytes (RAC) by about 20-30 fold (p<0.0001) when compared to the control (CON) group (middle panel). Treatment with BGE-105 (RAC+105) significantly reduced biomarker C3 expression by 10-20 fold (p=0.0015) as compared to the RAC group (RAC). FIG. 8 right panel shows IL-6 was upregulated in RAC-induced reactive astrocytes (RAC) by about 10-15 fold (p<0.0001). Treatment with BGE-105 (RAC+105) significantly reduced IL-6 expression by about 5-10 fold (p<0.0001).

FIG. 9 shows that BGE-105 reduced astrocytic release of CXCL1 and IL-6. FIG. 9 left panel shows treatment with BGE-105 (RAC+105, Group 4) greatly reduced astrocyte release of CXCL1, and thus the concentration of CXCL1 (p=0.0032). Reactive astrocytes (RAC, Group 3) increased release of CXCL1 by about 30000-40000 fold when compared to the control astrocytes (CON, Group 1) (p<0.0001). A similar result of RAC+BAY, Group 6 was observed for treatment with NF-κB inhibitor (RAC+BAY, p<0.0001). FIG. 9 right panel shows reactive astrocytes (RAC) treated with BGE-105 (RAC+BGE-105, Group 4) reduced astrocytic release of IL-6 and thus reduced the concentration of IL-6 (p=0.0008). Reactive astrocytes (RAC) increased release of IL-6 by about 1600-2400 fold when compared to the control astrocytes (CON) (p<0.0001). A Similar result of Group 6 (RAC+BAY) was observed for treatment with NF-κB inhibitor (RAC+BAY, p<0.0001).

FIGS. 10A-10D show that BGE-105 activates apelin receptor (APJ) signaling in astrocytes. Shown are APJ, p-AKT, t-AKT and β-actin protein expressions in control astrocyte cells, cells treated with reactive astrocyte cocktail (RAC), and RAC cells treated with BGE-105 (RAC+BGE-105 [50 nM]). FIG. 10A shows that the expression of APJ was comparable in all three experimental groups (Control (CON), RAC, RAC+BGE-105). No significant fold change in expression was detected when normalized with β-actin expression (FIG. 10B). FIG. 10C shows expression of p-AKT was increased in the RAC+BGE-105 group, while expression of t-AKT remained comparable in all three experimental groups. Expression level of p-AKT was significantly increased in response to RAC+BGE-105 treatment by about 2 folds, as compared to t-AKT, and normalized with β-actin expression (FIG. 10D). p-AKT: phosphorylated AKT.

FIGS. 11A-11D show that BGE-105 decreases cytokine release in a dose-dependent manner. FIG. 11A shows relative fold change in expression of the various cytokines. The legend on the right ranges from 0 fold change (blue) to 1-fold change (or no change, labeled in white), up to 4+ fold change (dark red). FIG. 11A shows reduction of a panel of cytokines and chemokines release that was detected in astrocytes treated with reactive astrocyte cocktail (RAC) and various doses of BGE-105 (RAC+BGE-105 [10 nM, 50 nM, 250 nM]) in comparison to RAC. FIG. 11B shows transcription factor enrichment of biomarkers: IKBKB, IRF1, STATE, NF-κB1, and RELA. FIG. 11C shows fold changes of cytokine and chemokine release detected from different treatments (RAC, RAC+105 (10 nM), RAC+105 (50 nM), RAC+105 (250 nM). FIG. 11C shows that there is a higher number of downregulated (or <1 fold change) cytokines as the dose of BGE-105 increases. Treatment with BGE-105 (RAC+BGE-105 [50 nM and 250 nM]) significantly reduced cytokine and chemokine release. FIG. 11D shows protein bands of cytokine and chemokine as quantified by western blot and presented in dot blots.

FIG. 12 shows treatment of reactive astrocytes with BGE-105 reduces astrocytic release of CXCL1 and IL-6. FIG. 12 left panel shows that astrocytes treated with reactive astrocyte cocktail (RAC) had increased concentration/release of CXCL1 by about 40000-60000 pg/ml as compared to the control (CON) group. BGE-105 (RAC+BGE-105 [50 nM, 250 nM]) significantly reduced CXCL1 concentration/release to about 40000 pg/ml (50 nM) or about 20000-40000 pg/ml (250 nM). It is noted that no significant difference of CXCL1 concentration/release was detected between the BGE-105 (RAC+BGE-105 [50 nM]) and apelin (APL) (RAC+APL [50 nM]) treatment groups. FIG. 12 right panel shows treatment with RAC increased concentration/release of IL-6 by about 10000 pg/ml as compared to the control (CON) group. BGE-105 (RAC+BGE-105 [50 nM, 250 nM]) significantly reduced IL-6 concentration/release to about 5000-10000 pg/ml (50 nM) or about 10000 pg/ml (250 nM). It is noted that no significant difference of IL-6 concentration/release was detected between the BGE-105 (RAC+BGE-105 [50 nM]) and apelin (APL) (RAC+APL [50 nM]) treatment groups.

FIGS. 13A-13C shows BGE-105 inhibits NF-κB activation and IκBα phosphorylation in astrocytes. FIG. 13A shows NF-κB, p-IκBα, t-IκBα, and β-actin protein expressions in control astrocyte cells, astrocytes treated with reactive astrocyte cocktail (RAC), and with BGE-105 (RAC+BGE-105 [50 nM]). FIG. 13B shows NF-κB p65 expression normalized to β-actin. RAC increased NF-κB p65 expression as compared to the control (CON).However, treatment with BGE-105 (RAC+BGE-105 (50 nM)) significantly reduced NF-κB p65 expression. FIG. 13C shows the ratio of p-IκBα/tIκBα expression normalized to β-actin. RAC had increased ratio of p-IκBα/tIκBα expression and treatment with BGE-105 (RAC+BGE105) significantly reduced the ratio of p-IκBα/tIκBα expression.

FIGS. 14A-14B show reactive astrocytes treated with BGE-105 had a dose-dependent effects on NF-κB signaling transcription response. FIG. 14A is a heat map illustrating expression of biomarkers associated with the NF-κB signaling pathway in astrocytes treated with reactive astrocyte cocktail (RAC), and in response to treatment of BGE-105 (RAC+BGE-105 [50 nM, 250 nM]). The legend shows fold change in expression compared to the control group (not shown), ranging from −10-fold change (blue) to 0-fold change (or no change, labeled in white), up to 30 fold change (dark red). FIG. 14B shows fold change in expression relative to control of exemplary subset of biomarkers from FIG. 14A: Rela, Il2, Cxcl3, and Csf3. Showing two cases of downregulation (Rela [decreased by 182%], Cxcl3 [decreased by 55%]) of proinflammatory genes and two cases of upregulation (Il2 increased by 184%], Csf3 [increased by 16%]) of anti-inflammatory/neurotrophic genes in response to treatment of reactive astrocytes with a higher dose of BGE-105 (250 nM) (RAC+BGE-105 [250 nM]).

FIG. 15 shows that BGE-105 improved glutamate clearance deficit in RAC astrocytes. Glutamate clearance was significantly decreased in RAC astrocytes (RAC), which may contribute to glutamate excitotoxicity in neurodegeneration/aging. BGE-105 (RAC+BGE-105 [10 nM, 50 nM, 250 nM]) and apelin (APL) (RAC+APL [50 nM]) significantly and statistically increased the percentage of glutamate uptake.

FIGS. 16A-16B show reactive astrocytes treated with BGE-105 improved cellular viability after RAC-conditioned media challenge. Shown is a percentage change in viability relative to RAC in astrocytes in control (RAC) and BGE-105 (RAC+BGE105 [50 nM, 250 nM]) and apelin (APL) (RAC+APL [50 nM]) treatment groups. FIG. 16A shows BGE-105 (RAC+BGE105 [250 nM]) significantly and statistically increased cellular viability by about 30% as compared to RAC. FIG. 16B shows exogenous BGE-105 (RAC+ex105 [50 nM]), where BGE-105 was added directly to neurons in addition to the toxic RAC media, did not provide statistical change of cell viability relative to RAC. Exogenous BGE-105 did not directly protect neurons against RAC-induced neurotoxicity.

### 5. DETAILED DESCRIPTION

5.1. Methods of Reducing BBB Permeability Using Apelin Receptor Modulators

As summarized above, an aspect of the present disclosure provides methods for reducing blood-brain barrier (BBB) permeability in a subject in need thereof using an apelin receptor modulator, e.g., using an apelin receptor agonist of particular structural class. In some embodiments, the methods of this disclosure provide for reducing BBB permeability in a subject, provide for restoring BBB permeability levels to a normal or healthy level, reducing BBB permeability, reducing BBB leakage, maintaining BBB intactness, or any combination thereof.

During aging, various mechanisms cause BBB breakdown and increase BBB permeability. Increased BBB permeability is associated with dementia and various neurodegenerative diseases. See e.g., Hussain et al. “Blood—Brain Barrier Breakdown: An Emerging Biomarker of Cognitive Impairment in Normal Aging and Dementia”, Front. Neurosci., 19 Aug. 2021 (doi.org/10.3389/fnins.2021.688090). Postoperative delirium is associated with a breakdown in the BBB. This increased permeability is dynamic and associated with a neuroinflammatory and lactate response. See e.g., Taylor, Jennifer et al., (“Postoperative delirium and changes in the blood—brain barrier, neuroinflammation, and cerebrospinal fluid lactate: a prospective cohort study” Neuroscience And Neuroanaesthesia, Volume 129, Issue 2, P219-230, August 2022).

The present inventors have shown that BGE-105 reversed BBB permeability in aged mice (26-month-old), demonstrating in vivo activity in a model of age-related cognitive impairment.

Systemic inflammation can also cause increased in BBB permeability and lead to a condition of impaired cognition, e.g., acute cognitive impairment. In some embodiments, the acute cognitive impairment is dementia, delirium or post-operative cognitive dysfunction. Such acute episodes of delirium can also induce injury and contribute to long-term cognitive decline. Additionally, peripheral inflammation is known to promote BBB leakage and cognitive impairment. Preliminary studies using BGE-105 on aged mice showed that BGE-105 decreased circulating levels of two cytokines (CXCL1/13) associated with mortality, neutrophil recruitment, and propagation of inflammation.

The present inventors further demonstrated that normal mice (12-month-old) treated with BGE-105 exhibit a significant reversal of LPS-induced BBB permeability, demonstrating in vivo activity in a model of acute cognitive impairment.

Thus, an exemplary apelin receptor modulator reduced BBB permeability in vivo in models of cognitive impairment, including acute or age-related cognitive impairment. These results indicate that apelin receptor modulators, such as BGE-105, would be effective in treating disorders associated with BBB Permeability. In some embodiments, the method of reducing BBB permeability treats acute delirium in a patient. In some embodiments, the method of reducing BBB permeability treats neurodegenerative disease or dementia in an aged patient. In some embodiments, reducing BBB permeability treats delirium due to trauma. In some embodiments, reducing BBB permeability treats delirium due to trauma from a hip fracture or cardiovascular surgery. delirium due to trauma, or delirium due to a surgical procedure. In some embodiments, reducing BBB permeability treats inflammation, such as, e.g., neuroinflammation or peripheral inflammation.

As described herein, BBB permeability can be assessed or determined over the course of treatment via a variety of direct and/or indirect methods. In some embodiments, a reduction in BBB permeability is determined by comparison to a normal or healthy level of BBB permeability, with change(s) assessed over time. In some embodiments, a reduction in BBB permeability is determined by comparison to a baseline increased level of BBB permeability that is assessed in the subject prior to treatment. In some embodiments, BBB permeability can be assessed indirectly via an assessment of one or more symptoms of cognitive impairment (e.g., as described herein).

5.1.1. Acute Cognitive Impairment Model

The present disclosure describes the assessment of apelin receptor agonists in vivo in a mouse model that generally relates to acute cognitive impairment, induced by inflammation.

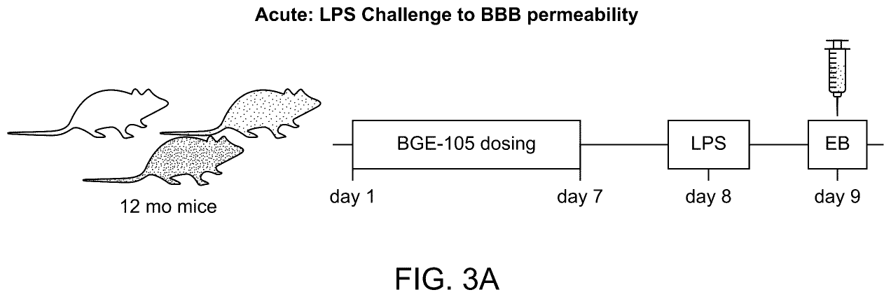

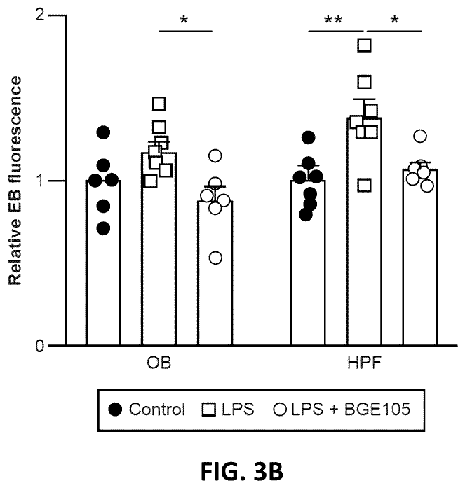

In a first set of experiments (see FIG. 3A), the activity of an exemplary apelin receptor agonist was assessed in 12-month-old mice challenged with LPS to induce an increase in BBB permeability in the olfactory bulb (OB) and hippocampus (HPF). FIG. 3B (control versus LPS). Further details are provided in Example 2 of the experimental section. The present inventors demonstrated that such mice treated with BGE-105 exhibit a significant reversal of the LPS-induced BBB permeability as compared to vehicle treated mice. FIG. 3B (LPS versus LPS+BGE105).

5.1.2. Cognitive Impairment Model in Aged Subjects

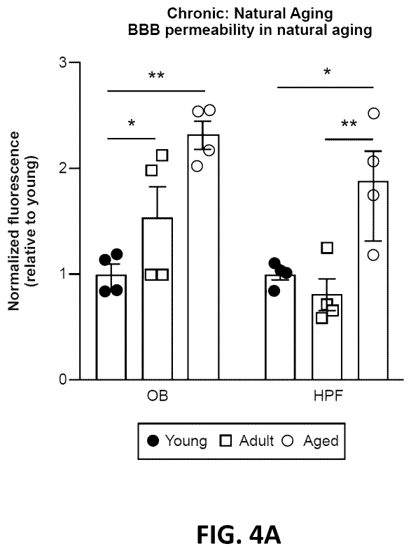

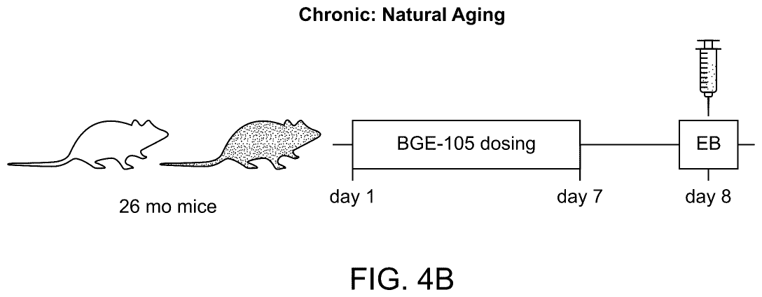

In a second set of experiments, the activity of an exemplary apelin receptor agonist was assessed in an aged mouse model for age-related increase in BBB permeability. FIG. 4A illustrates the increased BBB permeability in aged mice of the model.

The present inventors demonstrated that pretreatment of mice with BGE-105 significantly reversed age-induced increase in BBB permeability in the aged mice. See e.g., FIG. 4C, aged versus aged+BGE-105 in the olfactory bulb (OB) as compared to HPF (−28%) or cortical subplate (CTX) (data not shown). Further details are provided in Example 3 of the experimental section, demonstrating in vivo activity in a model of age-related cognitive impairment.

5.2. Methods of Treating a Disease or Disorder Associated with BBB Permeability

Accordingly, in one aspect the present disclosure provides a method of treating a disorder associated with BBB permeability, using an apelin receptor modulator.

The method includes administering to a subject in need thereof a therapeutically effective amount of an apelin receptor modulator of formula (I) or (II) (e.g., as described herein), such as BGE-105.

The “disorder associated with BBB permeability” (referred to interchangeably herein as an “BBB permeability-related disorder” and “disorder related to BBB permeability” refers to a disorder or condition that leads to, or is susceptible to increased BBB permeability and/or abnormal BBB permeability in a mammalian subject. In other words, a disorder associated with BBB permeability can include disorders or conditions that have an effect on BBB breakdown or BBB permeability. For example, such disorders can include one or more neurodegenerative diseases, including disorders that cause neurodegeneration, neuro-inflammation and/or cognitive impairment. In some embodiments, increased BBB permeability or abnormal permeability leads to an acute or chronic cognitive impairment.

In some embodiments, the disorder associated with BBB permeability is dementia. Dementia is not a specific disease but rather encompasses a variety of conditions characterized by an impaired cognitive ability and problems with memory, language, thinking or judgment that interferes with doing everyday activities. In some embodiments, the disorder is vascular dementia (VaD). Vascular dementia is a neurodegenerative disease characterized by the loss of cognitive function resulting from ischemic, ischemic-hypoxic, or hemorrhagic brain lesions as a result of cardiovascular diseases and cardiovascular pathologic changes. After AD, VaD is also considered the second most common type of dementia. The symptoms of VaD include cognitive loss, headaches, insomnia and memory loss.

In some embodiments, the disorder associated with BBB permeability is characterized by cognitive impairment. In some embodiments, the cognitive impairment is in subjects who are having, or at risk of developing, a neurodegenerative disease or an associated or related condition.

In some embodiments, the subject is exhibiting one or more symptoms of age-related cognitive impairment. Mild cognitive impairment (MCI) is the stage between the expected cognitive decline of normal aging and the more serious decline of dementia. Symptoms of MCI can remain stable for years, or in some cases progress to a dementia. Acute cognitive impairment can be associated with acute inflammation, such as inflammation associated with a surgery or other injury. In certain embodiments, the acute cognitive impairment is referred to, or characterized as, delirium. Delirium is a condition that affects the brain and can appear suddenly, within hours or days of an injury or other cause. Some characteristics of delirium include trouble focusing (inattention), sudden changes in behavior, and confusion. For most people, delirium is short-lived, usually only a few days.

In some embodiments, the disorder associated with BBB permeability is delirium. In some embodiments, the disorder associated with BBB permeability is post-operative delirium. In certain embodiments, the disorder associated with BBB permeability is intensive care unit (ICU) delirium. Patients in an intensive care unit (ICU) can be at risk of developing ICU delirium. In some cases, about two-thirds of ICU patients develop delirium, with those on breathing machines tending to be most at risk. ICU delirium should be diagnosed and treated as quickly as possible, as patients with ICU delirium can have poor outcomes if they do not receive treatment, leading to long-term problems, such as depression and anxiety.

In certain embodiments, the disorder associated with BBB permeability is postoperative cognitive dysfunction (POCD). POCD is a state in which a patient's memory and learning decline after surgery. All age groups of patients are at risk, although those over 60 years of age are more commonly affected by POCD. Symptoms that have been reported for POCD include: difficulty in remembering and recalling; inability to complete tasks that were previously not difficult; issues with intellectual performance; difficulty with multitasking; reduced psychomotor skills; language comprehension difficulties; and issues with social integration.

In some embodiments, the disorder associated with BBB permeability is inflammation. In some embodiments, the disorder associated with BBB permeability is neuroinflammation. In some embodiments, the disorder associated with BBB permeability is a disorder associated with the central nervous system (CNS). In some embodiments, the disorder associated with BBB permeability is peripheral inflammation.

In some embodiments, the disorder associated with BBB permeability is a neurodegenerative disease or an associated or related condition. Any neurodegenerative disease associated with changes in BBB permeability and function can be targeted for treatment according to the methods of this disclosure.

In certain embodiments, the neurodegenerative disease is Alzheimer's disease (AD). AD can be referred to as a neurodegenerative disease, and a type of dementia. In AD patients, the BBB shows leakages in brain vasculature, the perivascular aggregation of fibrinogen, albumin, thrombin, and immunoglobulin (IgG), the loss of TJs, and the degeneration of ECs and pericytes.

In certain embodiments, the neurodegenerative disease is Parkinson's disease (PD).

In certain embodiments, the neurodegenerative disease is amyotrophic lateral sclerosis (ALS).

In certain embodiments, the neurodegenerative disease is multiple sclerosis (MS).

In some embodiments, the disorder or condition is a brain injury. Brain injury, such as ischemic, hemorrhagic, or traumatic, can lead to dysfunction of the BBB. In certain embodiments, the disorder associated with BBB permeability is traumatic brain injury (TBI). The methods of this disclosure can provide for treatment of post-traumatic dysfunction of the BBB.

In some embodiments, the disorder associated with BBB permeability is stroke. In some embodiments, the disorder associated with BBB permeability is ischemic stroke. In some embodiments, the disorder associated with BBB permeability is a hemorrhage. In some embodiments, the disorder associated with BBB permeability is amyloid-beta induced memory deficits.

In certain embodiments, the disorder associated with BBB permeability is stroke. In certain embodiments, the disorder associated with BBB permeability is ischemic stroke. The methods of this disclosure can provide for treatment of post-stroke dysfunction of the BBB.

In certain embodiments, the disorder associated with BBB permeability is neuroinflammation. In some embodiments, the disorder associated with BBB permeability is peripheral inflammation. The methods of this disclosure can provide for treatment of neuroinflammation, such as peripheral inflammation.

5.3. Methods of treating Dementia

Accordingly, in one aspect the present disclosure provides a method of treating dementia in a subject, using an apelin receptor modulator.

In some embodiments, the method includes administering to a subject in need thereof a therapeutically effective amount of an apelin receptor modulator of formula (I) or (II) (e.g., as described herein), such as BGE-105.

In certain embodiments, dementia is acute dementia. In certain embodiments, dementia is chronic or progressive dementia. In certain embodiments, the subject has vascular dementia (VaD).

5.4. Methods of Treating Cognitive Impairment or Dysfunction

Accordingly, in one aspect the present disclosure provides a method of treating cognitive impairment or dysfunction in a subject, using an apelin receptor modulator.

In some embodiments, the method includes administering to a subject in need thereof a therapeutically effective amount of an apelin receptor modulator of formula (I) or (II) (e.g., as described herein), such as BGE-105.

In certain embodiments, the subject has acute, mild, or progressive cognitive impairment. In certain embodiments, the subject has post-operative cognitive dysfunction (POCD).

5.5. Methods of Treating Delirium

Accordingly, in one aspect the present disclosure provides a method of treating delirium in a subject, using an apelin receptor modulator.

In some embodiments, the method includes administering to a subject in need thereof a therapeutically effective amount of an apelin receptor modulator of formula (I) or (II) (e.g., as described herein), such as BGE-105.

In some embodiments, delirium is post-operative delirium. In certain embodiments, delirium is diagnosed in the patient following surgery (e.g., cardiovascular surgery). In certain embodiments, delirium is diagnosed after a bone fracture (e.g., hip fracture). In some embodiments, delirium is ICU induced delirium. In some embodiments, delirium is diagnosed due to trauma.

5.6. Methods of Reducing Neuroinflammation

Accordingly, in one aspect the present disclosure provides a method of reducing neuroinflammation in a subject, using an apelin receptor modulator.

In some embodiments, the method includes administering to a subject in need thereof a therapeutically effective amount of an apelin receptor modulator of formula (I) or (II) (e.g., as described herein), such as BGE-105.

5.7. Methods of Reducing Neurotoxicity Using Apelin Receptor Modulators

As summarized above, the present disclosure provides methods for reducing neurotoxicity or neurodegeneration in a subject in need thereof using an apelin receptor modulator, e.g., using an apelin receptor modulator.

In some embodiments, the method includes administering to a subject in need thereof a therapeutically effective amount of an apelin receptor modulator of formula (I) or (II) (e.g., as described herein), such as BGE-105.

In some embodiments, the methods of this disclosure provide for reducing neurotoxicity or neurodegeneration in a subject, provide for reducing neurotoxicity or neurodegeneration in astrocytic cells, reducing astrocyte cytokine or chemokine release, inhibiting or reducing NF-κB signaling transcriptional response, and/or restoring glutamate clearance deficiency to a normal or healthy level, or any combination thereof.

Astrocytes are primary cells expressing Apelin receptor (Apinr). BGE-105 is an agonist of the apelin receptor. The present inventors showed that apelin receptor is abundantly expressed and activated by BGE-105 in astrocytes and that BG5-105 dampens astrocytic inflammatory response following RAC exposure in mouse astrocyte cells. The present inventors show that BGE-105 also limits RAC-induced astrocyte inflammation through modifications of NF-κB signaling. BGE-105 improved cellular function in astrocytes and protected against RAC-induced cell death in neurons, indicating BGE-105 is effective in reducing neurotoxicity in degenerative or aging astrocytes and thus may be used for treating neurodegenerative diseases.

5.8. Methods of Treating a Neurodegenerative Disease or Disorder

Accordingly, in one aspect the present disclosure provides a method of treating a neurodegenerative disease or disorder in a subject, using an apelin receptor modulator.

In some embodiments, the method includes administering to a subject in need thereof a therapeutically effective amount of an apelin receptor modulator of formula (I) or (II) (e.g., as described herein), such as BGE-105.

In some embodiments, neurodegenerative disease selected from Alzheimer's disease (AD), Parkinson's disease (PD), amyotrophic lateral sclerosis (ALS), stroke, Huntington's disease (HD), and multiple sclerosis (MS). In some embodiments, the disorder is traumatic brain injury (TBI).

5.9. Patient Age

In some embodiments of the methods of the present disclosure, the subject is aged.

In some embodiments, the subject is human. When the subject is human, the subject can be referred to as a patient. In some embodiments, the patient is at least 40-years-old. In some embodiments, the patient is at least 50-years-old. In some embodiments, the patient is at least 60-years-old. In some embodiments, the patient is at least 65-years-old. In some embodiments, the patient is at least 70-years-old. In some embodiments, the patient is at least 75-years-old. In some embodiments, the patient is at least 80-years-old. In some embodiments, the patient is at least 85-years-old. In some embodiments, the patient is at least 90-years-old. In certain embodiments, the patient is 40-50 years old, 50-60 years old, 60-70 years old, 70-80 years old, or 80-90 years old.

5.10. Assessment of Patients

A subject can be identified as in need of treatment according to the methods of this disclosure, using a variety of different direct and/or indirect assessment methods. A reduction in BBB permeability can be determined by comparison to a baseline level, e.g., a level determined prior to treatment directly (e.g., via an imaging method) or indirectly (e.g., via assessment of an associated biomarker in a sample of the subject) by a suitable assessment method. In some embodiments, a reduction in BBB permeability is achieved by practicing the methods of this disclosure is a 10% or more reduction, such as 20% or more, 30% or more, 40% or more, or 50% or more reduction in a baseline BBB permeability, as determined by a suitable direct or indirect assessment method.

A reduction in neurotoxicity associated with neurodegenerative diseases can be determined by comparison to a baseline level, e.g., a level determined prior to treatment directly (e.g., via an imaging method) or indirectly (e.g., via assessment of an associated biomarker in a sample of the subject) by a suitable assessment method. In some embodiments, a reduction in neurotoxicity associated with neurodegenerative diseases is achieved by practicing the methods of this disclosure is a 10% or more reduction, such as 20% or more, 30% or more, 40% or more, or 50% or more reduction in a baseline baseline neurotoxicity, as determined by a suitable direct or indirect assessment method.

BBB permeability and dysfunction or neurodegenerative diseases including related conditions can be assessed using a variety of methods, such as assessment by an imaging technique or electroencephalogram. See e.g., WO2021053684. Various other methods can be used to identify biomarkers associated with BBB breakdown and increased BBB permeability in a subject. See, e.g., Hussain et al. 2021. Blood-Brain Barrier Breakdown: An Emerging Biomarker of Cognitive Impairment in Normal Aging and Dementia. Front Neurosci. 2021 Aug. 19; 15:688090. Other methods for identifying and assessing neurodegenerative diseases include the combination of more detailed clinical assessments encompassing specific cognitive and neurophysiological testing, in addition to imaging, biochemical and genomic profiling. See, e.g., Henley et al., 2005. Biomarkers for neurodegenerative diseases. Current Opinion in Neurology 18(6):p 698-705, December 2005.

In some embodiments, BBB permeability, neurotoxicity, neurodegeneration, and/or cognitive impairment of the subject is assessed after the dosing. In some embodiments, the assessment is at least one day after dosing. In some embodiments, the assessment is at least one week after dosing. In some embodiments, the assessment is at least one month after dosing.

In certain embodiments, the subject is human and is undergoing mechanical ventilation (e.g. is mechanically ventilated at time of diagnosis). In some embodiments, the subject is human and is on a ventilator.

In some embodiments, the patient is on bedrest. In some embodiments, the patient is on a ventilator.

In some embodiments, the human subject has, or is identified as having, cognitive impairment (e.g., acute, mild, severe cognitive impairment). In some embodiments, the human subject has, or is identified as having, post-operative cognitive dysfunction. In some embodiments, the human subject has, or is identified as having, dementia. In some embodiments, the human subject has, or is identified as having, delirium (e.g., post-operative delirium, ICU induced delirium). Cognitive impairment can be assessed using a variety of diagnostic methods.

In some embodiments, the patient has, or is identified as having, increased neurotoxicity. In some embodiments, the patient has, or is identified as having, neuroinflammation. In certain embodiments, the neuroinflammation is peripheral inflammation. In some embodiments, the human subject has, or is at risk of developing, neurodegeneration.

In some embodiments, the patient has, or is identified as having, a neurodegenerative disease or condition selected from: Alzheimer's disease (AD), Parkinson's disease (PD), amyotrophic lateral sclerosis (ALS), stroke, Huntington's disease (HD), multiple sclerosis (MS), and traumatic brain injury (TBI).

In certain embodiments, the human subject has, or is at risk of developing, Alzheimer's disease (AD).

In certain embodiments, the human subject has, or is at risk of developing, Parkinson's disease (PD).

In certain embodiments, the human subject has, or is at risk of developing, amyotrophic lateral sclerosis (ALS).

In certain embodiments, the human subject has, or is at risk of developing, multiple sclerosis (MS).

In certain embodiments, the human subject has, or is at risk of developing, Huntington's disease (HD).

In certain embodiments, the human subject has, or is at risk of developing, neuroinflammation. In certain embodiments, the human subject has, or is at risk of developing, peripheral inflammation.

In some embodiments, the human subject has, or is at risk of developing, a stroke. In some embodiments, the human subject has, or is at risk of developing, a hemorrhage.

In some embodiments, the human subject has, or is at risk of developing, hyperplasticity (e.g., hyperplasticity associated with a neurodegenerative disease). In some embodiments, the human subject has, or is at risk of developing innate immune activation (e.g., associated with a neurodegenerative disease). In some embodiments, the human subject has, or is at risk of developing, blood—brain barrier dysfunction.

5.11. Apelin Receptor Modulators

Apelin is the endogenous ligand for the apelin receptor (also referred to as APJ, or APLNR). The apelin receptor is a member of the rhodopsin-like G protein-coupled receptor (GPCR) family. The apelin/APJ system is distributed in diverse periphery organ tissues and can play various roles in the physiology and pathophysiology of many organs. The apelin/APJ system participates in various cell activities such as proliferation, migration, apoptosis or inflammation. An apelin receptor modulators can activate the APJ system directly or indirectly, competitively, or non-competitively.

The suitability of an apelin receptor modulator for the treatment of a disorder, neurodegenerative disease, or condition associated with increased or abnormal BBB permeability according to the methods of this disclosure can be assessed in any of a number of animal models for neurodegenerative disease and/or BBB permeability. Animal models, for example, for Huntington's disease (see, e.g., Mangiarini et al., 1996, Cell 87: 493-506, Lin et al., 2001, Hum. Mol. Genet. 10: 137-144), Alzheimer's disease (Hsiao, 1998, Exp. Gerontol, 33: 883-889; Hsiao et al., 1996, Science 274: 99-102), Parkinson's disease (Kim et al., 2002, Nature 418: 50-56), and amyotrophic lateral sclerosis (Zhu et al., 2002, Nature 417: 74-78).

As further described below, in some embodiments of the methods of this disclosure, the apelin receptor modulator (e.g., apelin receptor agonist) is a compound described in U.S. Pat. Nos. 9,573,936 or 9,868,721, the disclosures of which are herein incorporated by reference in their entirety.

As known by those skilled in the art, certain compounds of this disclosure may exist in one or more tautomeric forms. Because one chemical structure may only be used to represent one tautomeric form, it will be understood that for convenience, referral to a compound of a given structural formula includes tautomers of the structure represented by the structural formula.

In some embodiments, the apelin receptor modulator is a compound of formula (I) or (II):

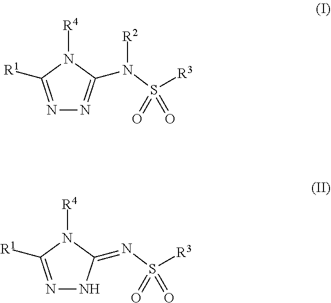

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —O(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

In some embodiments, the apelin receptor modulator is a compound of formula (I) or (II):

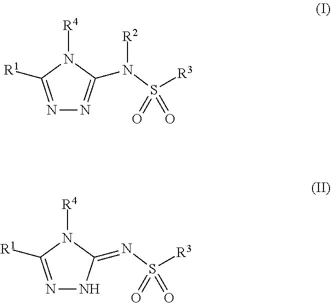

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆alkyl)₂, phenyl, C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, or S;
R² is selected from —H, or C₁-C₄ alkyl or is absent in the compounds of Formula II;
R³ is a group of formula —(CR^{3d}R^{3e})(CR^{3f}R^{3g})-Q;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), or —N(C₁-C₆alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), or —N(C₁-C₆alkyl)₂;
Q is a monocyclic or bicyclic C₆-C₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆alkyl)₂, —C(═O)—(C₁-C₆ alkyl), —C(═O)OH, —O(═O)—O—(C₁-C₆ alkyl), —C(═O)NH₂, —C(═O)NH(C₁-C₆ alkyl), —C(═O)N(C₁-C₆alkyl)₂, —S(═O)₂—(C₁-C₆ alkyl), phenyl, or a heteroaryl group, and the Q heterocyclyl group may be substituted with 1 oxo substituent;
R⁴ is selected from a monocyclic or bicyclic C₆-C₀ aryl group, a monocyclic or bicyclic heteroaryl group with 5 to 10 ring members containing 1, 2, or 3 heteroatoms independently selected from N, O, or S, or a monocyclic or bicyclic heterocyclyl group with 5 to 10 ring members containing 1, 2, 3, or 4 heteroatoms independently selected from N, O, or S, wherein the C₆-C₁₀ aryl group, the heteroaryl group, or the heterocyclyl group are unsubstituted or are substituted with 1, 2, or 3 R^{4a} substituents; and
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), or —C(═O)N(C₁-C₆ alkyl)₂, and the heterocyclyl R⁴ group may be further substituted with 1 oxo substituent.

As noted above, apelin receptor agonist compounds of this disclosure may exist in multiple tautomeric forms. This is particularly true in compounds of Formula I where R² is H. These forms are illustrated below as Tautomer A and Tautomer B:

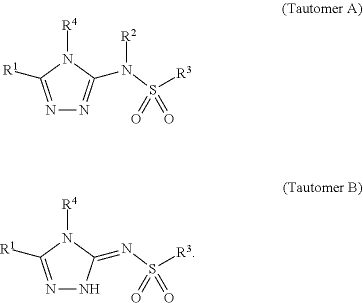

Apelin receptor agonist compounds of this disclosure are depicted structurally and generally named as compounds in the “Tautomer A” form. However, it is specifically contemplated and known that the compounds exist in “Tautomer B” form and thus compounds in “Tautomer B” form are expressly considered to be part of this disclosure. For this reason, the claims refer to compounds of Formula I and Formula II. Depending on the compound, some compounds may exist primarily in one form more than another. Also, depending on the compound and the energy required to convert one tautomer to the other, some compounds may exist as mixtures at room temperature whereas others may be isolated in one tautomeric form or the other.

In some embodiments of formula (I) and (II), le is an unsubstituted pyridyl or is a pyridyl substituted with 1 or 2 R^{1a} substituents.

In some embodiments of formula (I) and (II), lea in each instance is independently selected from —CH₃, —CH₂CH₃, —F, —Cl, —Br, —CN, —CF₃, —CH═CH₂, —C(═O)NH₂, —C(═O)NH(CH₃), —C(═O)N(CH₃)₂, —C(═O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula

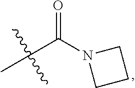 [SMILES: *C(=O)N1CCC1]

wherein the symbol [symbol] , when drawn across a bond, indicates the point of attachment to the rest of the molecule.

In some embodiments of formula (I) and (II), R¹ is selected from

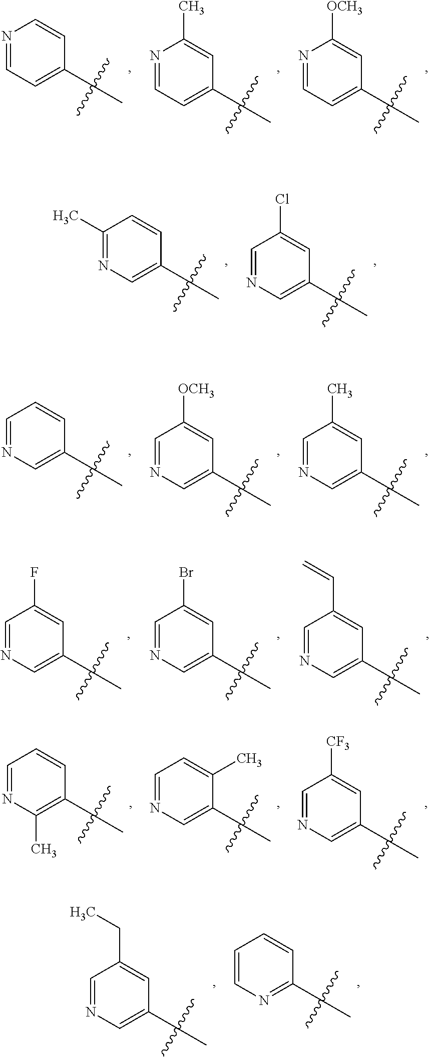

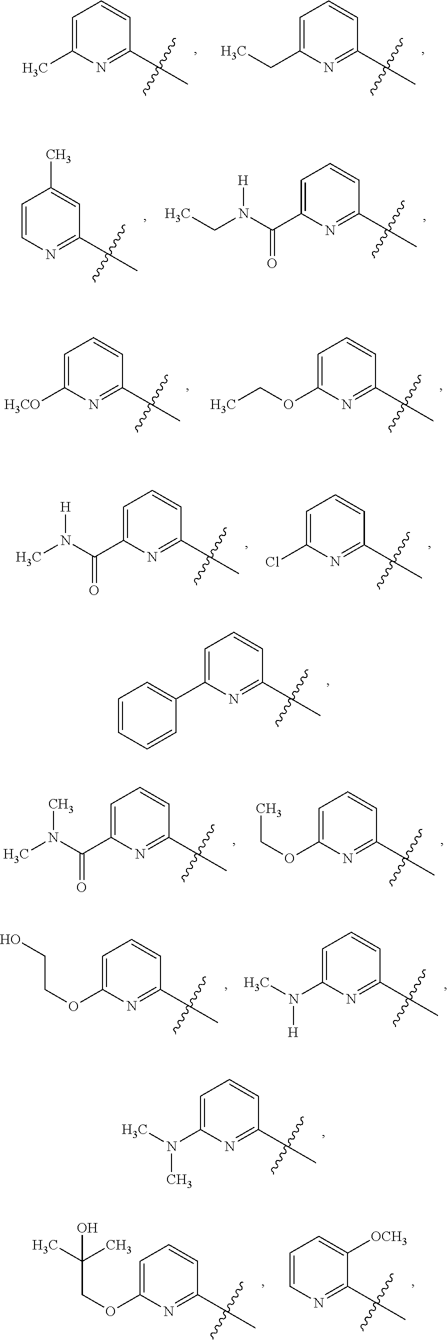

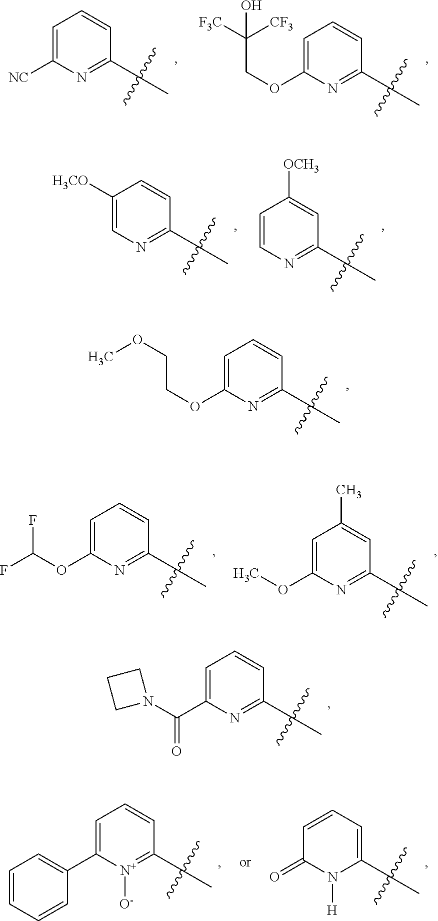

wherein the symbol [symbol] , when drawn across a bond, indicates the point of attachment to the rest of the molecule.

In some embodiments of formula (I) and (II), R¹ is selected from

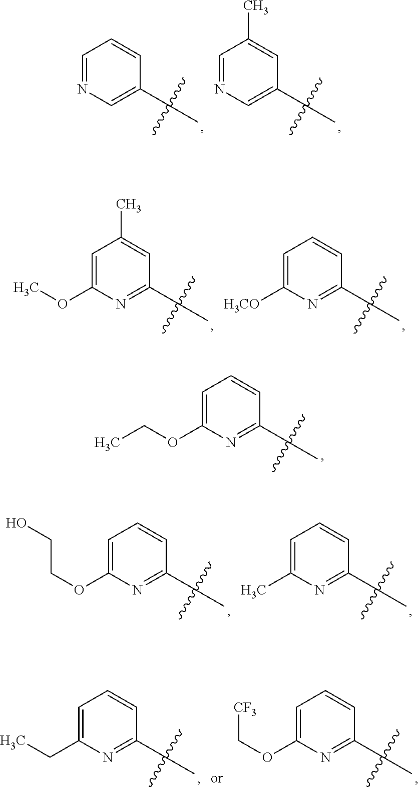

wherein the symbol [symbol] , when drawn across a bond, indicates the point of attachment to the rest of the molecule.

In some embodiments of formula (I) and (II), R² is —H.

In some embodiments of formula (I) and (II), R⁴ is a phenyl, pyridyl, pyrimidinyl, isoxazolyl, indolyl, naphthyl, or pyridinyl any of which may be unsubstituted or substituted with 1, 2, or 3 R^{4a} substituents. In some embodiments of formula (I) and (II), R⁴ is a phenyl substituted with 1 or 2 R^{4a} substituents. In some embodiments of formula (I) and (II), the 1 or 2 R^{4a} substituents are —O—(C₁-C₂ alkyl) groups.

In some embodiments of formula (I) and (II), R^{4a} is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.

In some embodiments of formula (I) and (II), R⁴ is selected from:

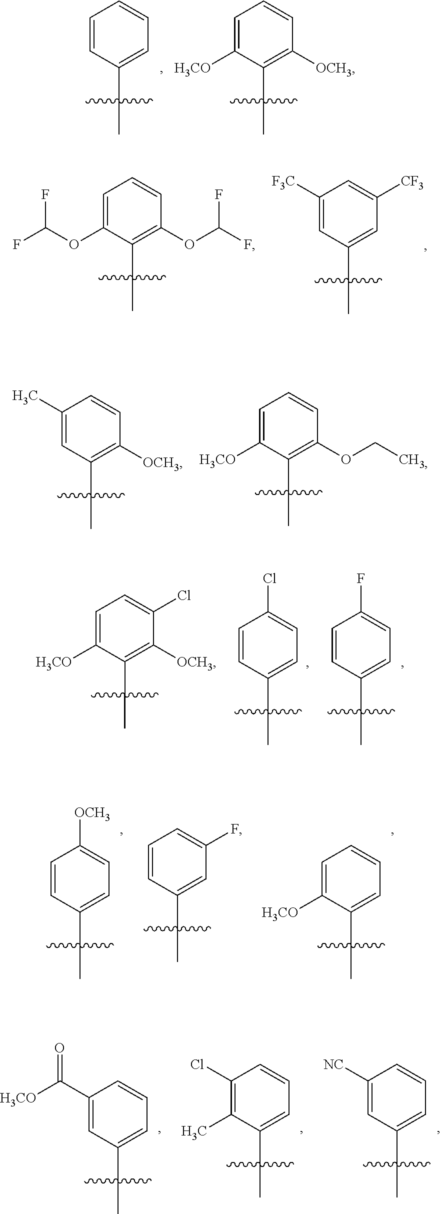

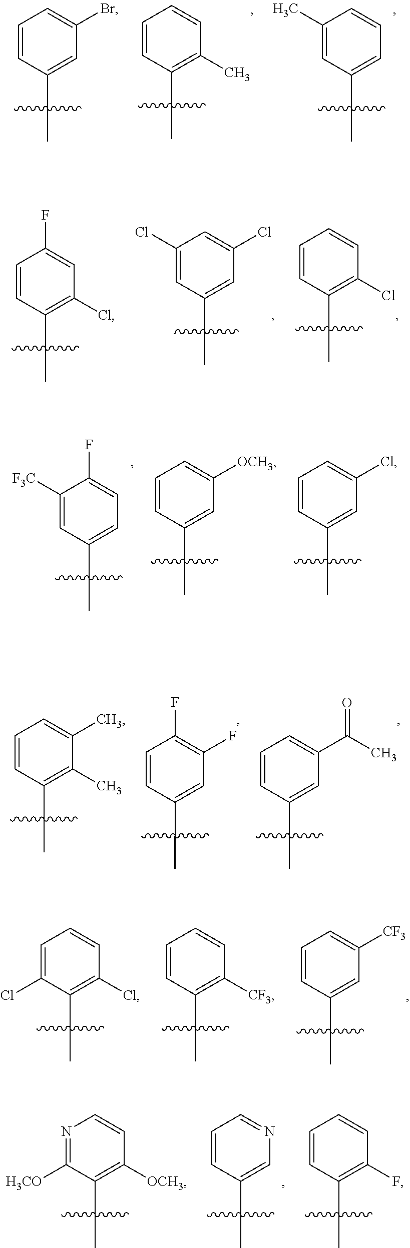

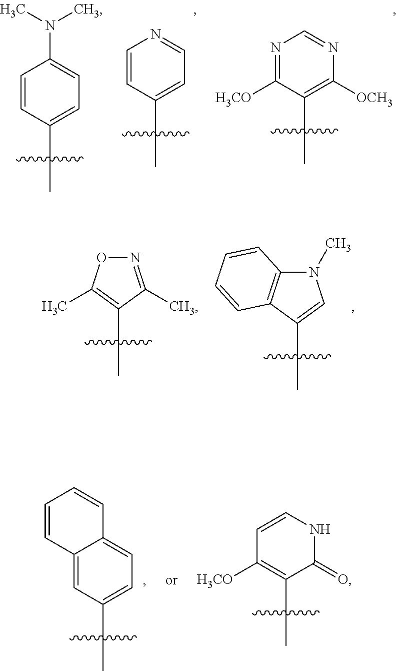

wherein the symbol [symbol] , when drawn across a bond, indicates the point of attachment to the rest of the molecule.

In some embodiments of formula (I) and (II), R³ is selected from a group of formula (CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyridinyl, pyrazolyl, or oxetanyl any of which may be unsubstituted or substituted with 1, 2, or 3, R^{Q} substituents.

In some embodiments of formula (I) and (II), Q is a monocyclic heteroaryl group with 5 or 6 ring members containing 1 or 2 heteroatoms selected from N, O, or S and Q is unsubstituted or is substituted with 1 or 2 R^{Q} substituents.

In some embodiments of formula (I) and (II), Q is selected from

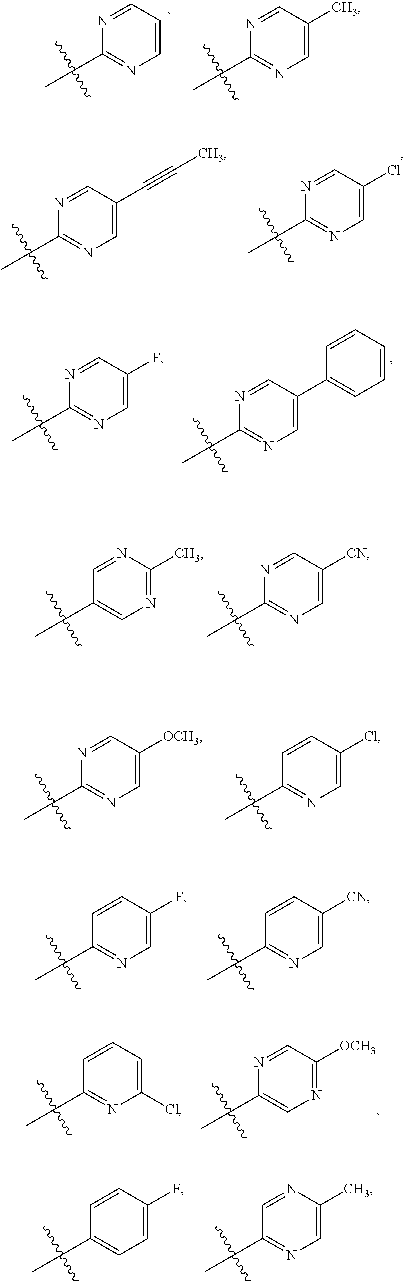

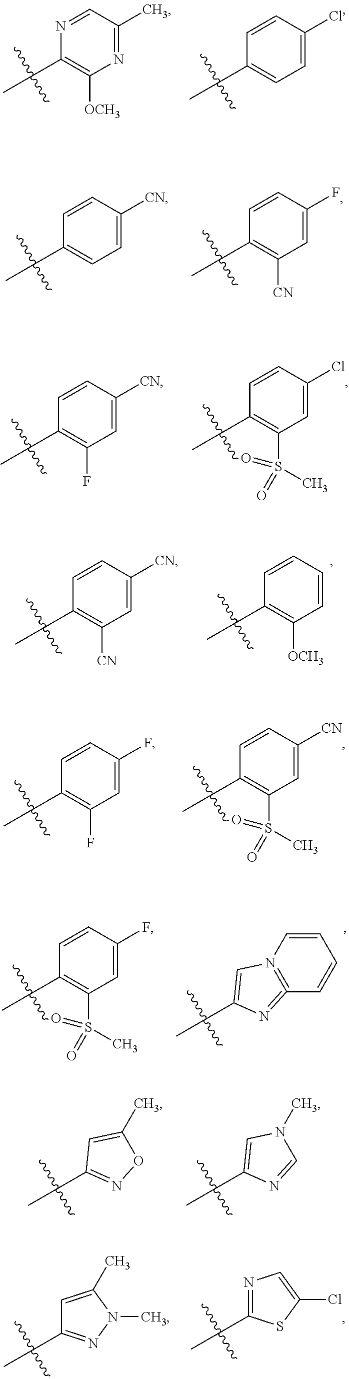

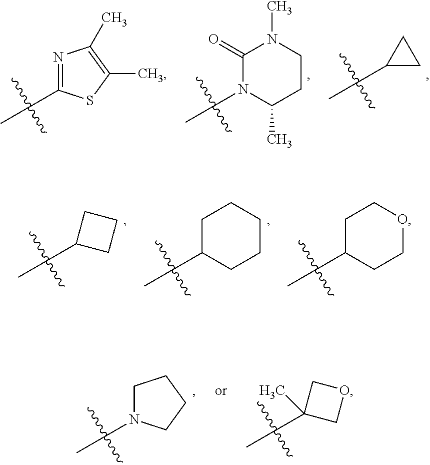

wherein the symbol [symbol] , when drawn across a bond, indicates the point of attachment to the rest of the molecule.

In some embodiments of formula (I) and (II), R³ is a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), R³ is a group of formula —(CR^{3d}R^{3e})—(CR^{3f}R^{3g})-Q.

In some embodiments of formula (I) and (II), R³ has the formula

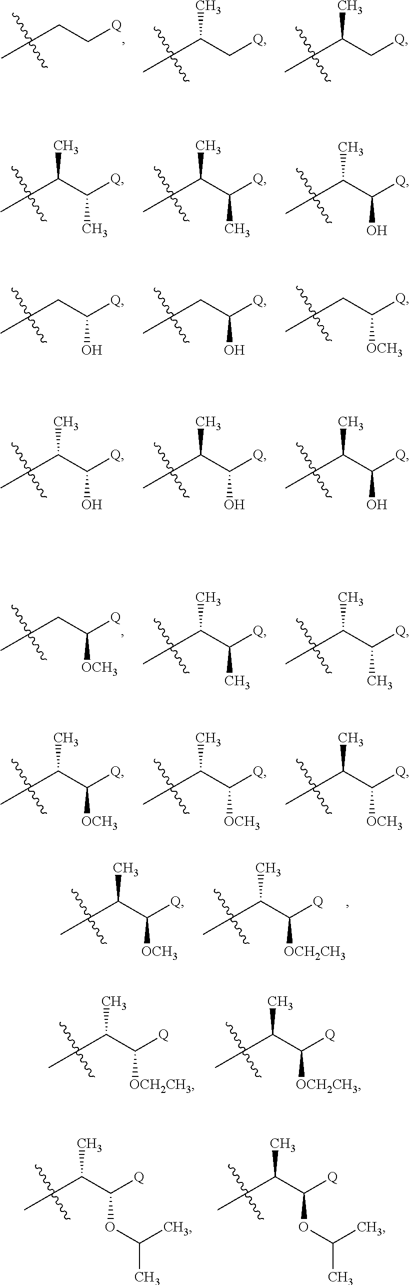

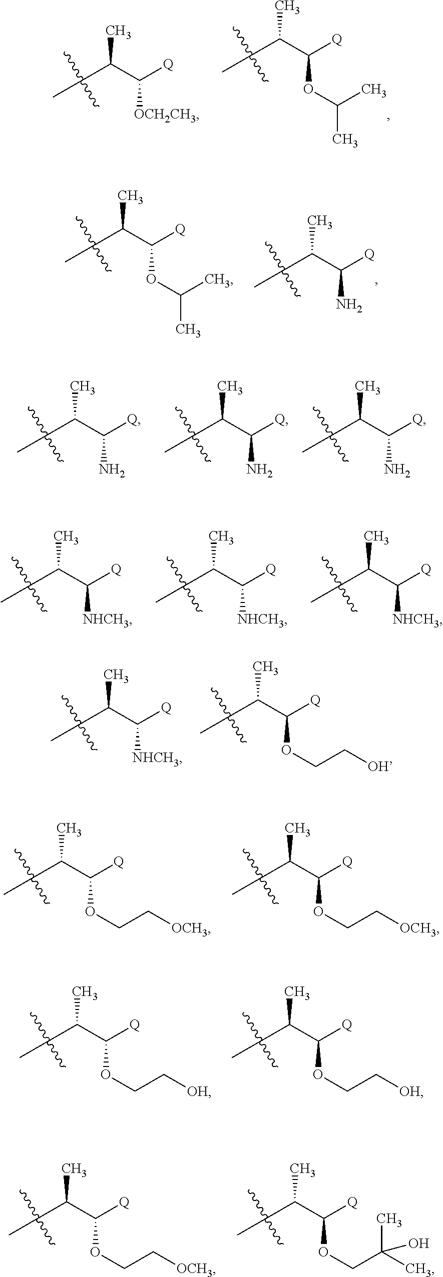

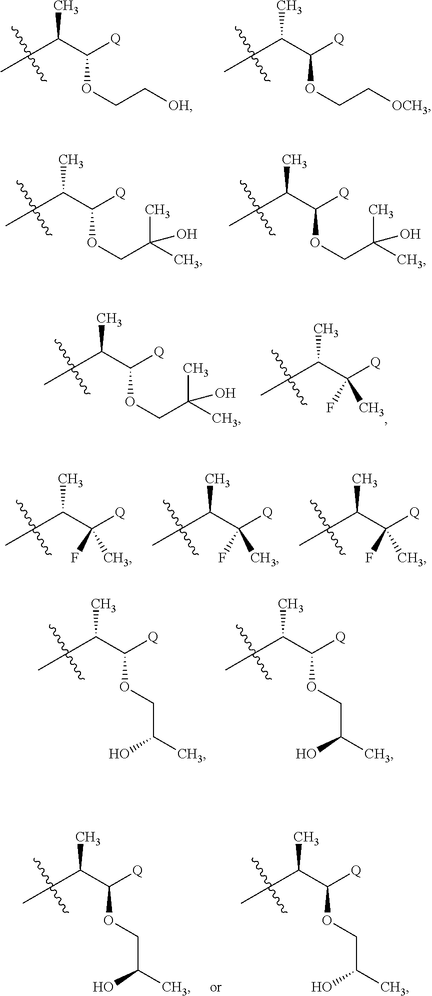

wherein the symbol [symbol] , when drawn across a bond, indicates the point of attachment to the rest of the molecule.

In some embodiments of formula (I) and (II), R³ has the formula

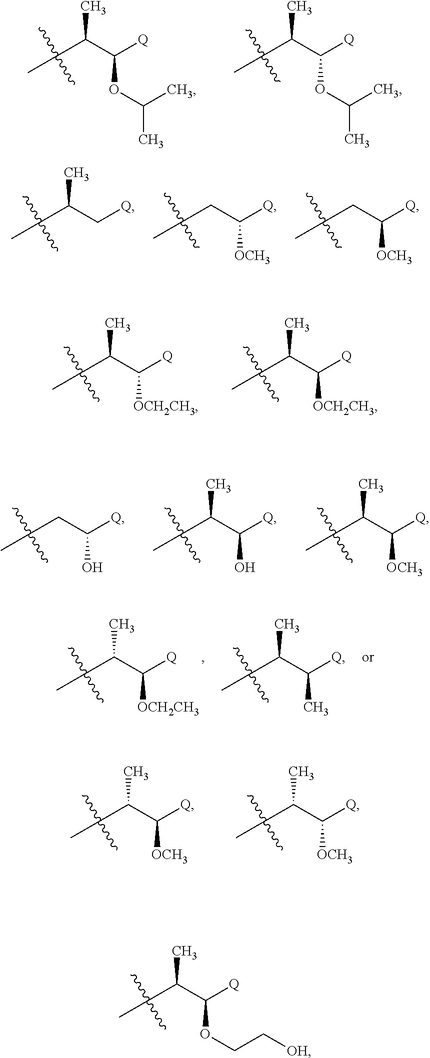

wherein the symbol [symbol] , when drawn across a bond, indicates the point of attachment to the rest of the molecule.

In particular embodiments of formula (I) and (II), the apelin receptor agonist is
(1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide;
(2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide;
(1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide;
(1S,2R)-1-(5-chloro-2-pyrimidinyl)-N-(4-(2,6-dim eth oxyphenyl)-5-(5-methyl-3-pyridinyl)-4H-1,2,4-triazol-3-yl)-1-methoxy-2-propanesulfonamide;
(1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide;
(1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide;
(1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide;
(2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide;
(1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide;
(1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide;
(1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide;
(1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide;
(1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide;
(2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide;
(1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide;
(1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide;
(1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide;
(1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide;
(2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide;
(1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide;
(1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide;
(1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide; or
(1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide.

In a particular embodiment of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide or the pharmaceutically acceptable salt thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propane sulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1 S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide or the pharmaceutically acceptable salt thereof.

In a particular embodiment of formula (I) and (II), the apelin receptor agonist is (2 S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide or the pharmaceutically acceptable salt thereof.

In a particular embodiment of formula (I) and (II), the apelin receptor agonist is (2 S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-difluorophenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(4,6-dimethoxy-5-pyrimidinyl)-5-(2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is Compound 2:

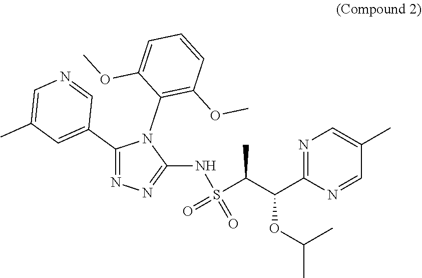

or a pharmaceutically acceptable salt thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide (BGE-105) or a pharmaceutically acceptable salt thereof.

In a particular embodiment of formula (I) and (II), the apelin receptor agonist is Compound 3

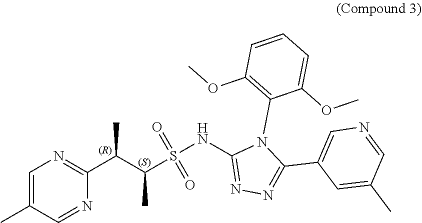

(BGE-105) or a pharmaceutically acceptable salt thereof.

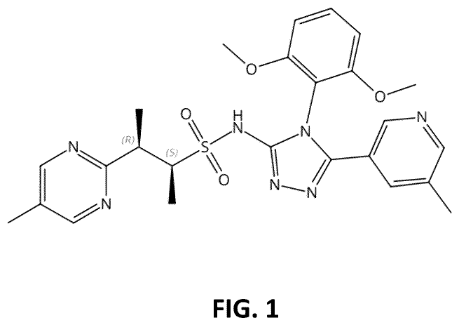

BGE-105 has the structure shown above and in FIG. 1. BGE-105 is known to activate the apelin receptor and induce a cardiovascular response in rats (Ason et al., JCI Insight. 5(8):1-16(2020)). Clinical trials were also done with BGE-105 to study the safety, tolerability, and pharmacokinetics in healthy subjects and those with suffering impaired renal function (NCT03318809) or heart failure (NCT03276728).

U.S. Pat. Nos. 9,573,936, 9,868,721, 9,745,286, 9,656,997, 9,751,864, 9,656,998, 9,845,310, 10,058,550, 10,221,162, and 10,344,016, the disclosures of which are incorporated herein by reference in their entirety, describe apelin receptor agonists of formula (I) or (II), and methods of synthesizing such triazole agonists of the apelin receptor, including BGE-105. See e.g., Example 263.0 of U.S. Pat. No. 9,573,936.

If any variable occurs more than one time in a chemical formula, its definition on each occurrence is independent of its definition at every other occurrence. If the chemical structure and chemical name conflict, the chemical structure is determinative of the identity of the compound. The compounds of this disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Certain compounds of this disclosure may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when R⁴ is a phenyl group and is substituted with two groups bonded to the C atoms adjacent to the point of attachment to the N atom of the triazole, then rotation of the phenyl may be restricted. In some instances, the barrier of rotation is high enough that the different atropisomers may be separated and isolated.

Unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed.

Various compounds of this disclosure contain one or more chiral centers, and can exist as racemic mixtures of enantiomers, mixtures of diastereomers or enantiomerically or optically pure compounds. This invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents.

Compounds of the present disclosure include, but are not limited to, compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. The term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

The term “solvate” refers to the compound formed by the interaction of a solvent and a compound. Suitable solvates are pharmaceutically acceptable solvates, such as hydrates, including monohydrates and hemi-hydrates.

The compounds of this disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., assay reagents, and diagnostic agents, e.g., in vivo imaging agents. All isotopic variations of the compounds of the invention, whether radioactive or not, are intended to be encompassed within the scope of the invention. For example, if a variable is said or shown to be H, this means that variable may also be deuterium (D) or tritium (T).

The term “pharmaceutically acceptable salt” refers to a salt that is acceptable for administration to a subject. Examples of pharmaceutically acceptable salts include, but are not limited to: mineral acid salts such as hydrochloride, hydrobromide, hydroiodide, phosphate, sulfate, and nitrate; sulfonic acid salts such as methanesulfonate, ethanesulfonate, benzenesulfonate, p-toluenesulfonate, and trifluoromethanesulfonate; organic acid salts such as oxalate, tartrate, citrate, maleate, succinate, acetate, trifluoroacetate, benzoate, mandelate, ascorbate, lactate, gluconate, and malate; amino acid salts such as glycine salt, lysine salt, arginine salt, ornithine salt, glutamate, and aspartate; inorganic salts such as lithium salt, sodium salt, potassium salt, calcium salt, and magnesium salt; and salts with organic bases such as ammonium salt, triethylamine salt, diisopropylamine salt, and cyclohexylamine salt. The term “salt(s)” as used herein encompass hydrate salt(s).

Other examples of pharmaceutically salts include anions of the compounds of the present disclosure compounded with a suitable cation. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions and methods that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions and methods that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Furthermore, if the compounds of the present disclosure or salts thereof form hydrates or solvates, these are also included in the scope of the compounds of the present disclosure or salts thereof.

Compounds included in the present compositions and methods that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

5.12. Pharmaceutical Composition

The apelin receptor agonist compounds used in the methods described herein can be formulated in any appropriate pharmaceutical composition for administration by any suitable route of administration. The pharmaceutical compositions can include the compound or the pharmaceutically acceptable salt thereof, the tautomer thereof, the pharmaceutically acceptable salt of the tautomer, the stereoisomer of any of the foregoing, or the mixture thereof according to any one of the embodiments described herein and at least one pharmaceutically acceptable excipient, carrier or diluent. In some such embodiments, the compound or the pharmaceutically acceptable salt thereof, the tautomer thereof, the pharmaceutically acceptable salt of the tautomer, the stereoisomer of any of the foregoing, or the mixture thereof according to any one of the embodiments is present in an amount effective for the treatment of a muscle condition (e.g., as described herein), for activating the APJ receptor.

Suitable routes of administration include, but are not limited to, oral, topical, and intravenous routes of administration. Suitable routes of administration also include intrathecal administration, such as via an injection into the spinal canal of the subject, or into the subarachnoid space. Suitable routes also include pulmonary administration, including by oral inhalation. The most suitable route may depend upon the condition and disorder of the recipient. The formulations may conveniently be presented in unit dosage form and may be prepared by any of the methods known in the art of pharmacy.

In some embodiments, the pharmaceutical composition is formulated for oral delivery whereas in other embodiments, the pharmaceutical composition is formulated for intravenous delivery. In some embodiments, the pharmaceutical composition is formulated for oral administration once a day or QD, and in some such formulations is a tablet where the effective amount of the active ingredient ranges from 5 mg to 60 mg, from 6 mg to 58 mg, from 10 mg to mg, from 15 mg to 30 mg, from 16 mg to 25 mg, or from 17 mg to 20 mg. In some such compositions, the amount of active ingredient is 17 mg. In some embodiments, the pharmaceutical composition is formulated for P.O administration once a day, where the effective amount of the active ingredient ranges from 5 mg to 300 mg, from 6 mg to 58 mg, from 10 mg to mg, from 15 mg to 30 mg, from 16 mg to 25 mg, or from 17 mg to 20 mg. In some such compositions, the amount of active ingredient is 17 mg.

All methods include the step of bringing into association an apelin agonist, or a salt thereof, with the carrier which constitutes one or more excipients. In general, the formulations are prepared by uniformly and intimately bringing into association the active ingredient with liquid carriers or finely divided solid carriers or both and then, if necessary, shaping the product into the desired formulation.

In certain embodiments, the route of administration for use in the methods described herein is parenteral administration. In certain embodiments, the route of administration for use in the methods described herein is intravenous administration (e.g., intravenous infusion). In certain embodiments, the route of administration for use in the methods described herein is oral administration. In certain embodiments, the route of administration for use in the methods described herein is constant intravenous infusion.

Formulations of the present methods suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain antioxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The pharmaceutical composition may comprise one or more pharmaceutical excipients. Any suitable pharmaceutical excipient may be used, and one of ordinary skill in the art is capable of selecting suitable pharmaceutical excipients. Pharmaceutical excipients include, for example, those described in the Handbook of Pharmaceutical Excipients, 8th Revised Ed. (2017).

5.13. Dosage Regimens

In various embodiments, the apelin receptor agonist (e.g., as described herein) is administered at a dose sufficient to reduce BBB permeability and/or treat a disorder associated with increased BBB permeability (e.g., as described herein), and/or treat neurodegenerative diseases such as increased neurotoxicity as described herein.

In various embodiments, the apelin receptor agonist (e.g., as described herein) is administered to an elderly subject in need thereof. In some embodiments, the elderly subject is human and at least 40 years old, at least 50 years old, at least 55 years old, at least 60-years-old, at least 65 years old, at least 70 years old, at least 75 years old, or at least 80 years old.

In various embodiments, the dose of the apelin receptor agonist is at least 0.01 mg/kg, such as at least 0.5 mg/kg, or at least 1 mg/kg. In certain embodiments, the dose is 25 mg/kg to 1,000 mg/kg per day.

In some embodiments, the apelin receptor agonist is administered in a dose that is independent of patient weight or surface area (flat dose).

In various embodiments, the dose is 1-5000 mg. In various embodiments, the dose is 25-2000 mg. In some embodiments, the dose is at least 60 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 420 mg, at least 440 mg, at least 460 mg, at least 480 mg, at least 500 mg, at least 520 mg, at least 550 mg, at least 580 mg, at least 600 mg, at least 650 mg, at least 700 mg, at least 750 mg, at least 800 mg, at least 850 mg, at least 900 mg, at least 950 mg, at least 1000 mg, at least 1100 mg, at least 1200 mg, at least 1300 mg, at least 1400 mg, or at least 100 mg. In various embodiments, the dose is 25-2000 mg. In some embodiments, the dose is at least 200 mg.

The apelin receptor agonist can be administered in a single dose or in multiple doses.

In some embodiments, the dose is administered daily.

In some embodiments, the dose is administered as a plurality of equally or unequally divided sub-doses.

In certain embodiments, the dose is administered continuously (e.g., IV infusion) for a period of time. In certain embodiments, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours). In certain embodiments, following the dose, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a dose and a 24 hour or 48-hour washout period, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a first dose and a 24 hour or 48-hour washout period, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours), followed by a second dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours).

In some embodiments, the apelin receptor agonist is administered orally, intravenously, intranasally, or intramuscularly. In some embodiments, the apelin receptor agonist is administered orally. In some embodiments, the apelin receptor agonist is administered intrathecally. In some embodiments, the apelin receptor agonist is administered intravenously. In some embodiments, the subject is a human patient on a ventilator.

In some embodiments, the apelin receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the apelin receptor agonist is administered continuously for at least 10 minutes, at least 20 minutes, at least 30 minutes, at least 40 minutes, at least 50 minutes, at least 1 hour, at least 2 hours, at least 3 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 48 hours, at least 72 hours, at least 100 hours, at least 110 hours, at least 115 hours, at least 120 hours, or at least 125 hours.

5.14. Dosage Form

In some embodiments, an apelin receptor modulator or salt thereof is administered in a suspension. In other embodiments, an apelin receptor modulator or salt thereof is administered in a solution. In some embodiments, an apelin receptor modulator or salt thereof is administered in a solid dosage form. In particular embodiments, the solid dosage form is a capsule. In particular embodiments, the solid dosage form is a tablet. In specific embodiments, an apelin receptor modulator is in a crystalline or amorphous form. In particular embodiments, an apelin receptor modulator is in amorphous form. In some embodiments, the apelin receptor modulator is an apelin receptor agonist.

In one aspect of the methods, the apelin receptor modulator, or the pharmaceutical composition including same, is administered intravenously, topically, orally, by inhalation, by infusion, by injection, intraperitoneally, intramuscularly, subcutaneously, intra-aurally, by intra-articular administration, by intra-mammary administration, by topical administration or by absorption through epithelial or mucocutaneous linings. In certain embodiments, the apelin receptor modulator, or the pharmaceutical composition including same, is administered via intravenous infusion.

5.15. Definitions

Unless defined otherwise, all technical and scientific terms used herein have the meaning commonly understood by a person skilled in the art to which this disclosure belongs.

The terms “individual,” “host,” and “subject” are used interchangeably, and refer to an animal to be treated, including but not limited to humans and non-human primates; rodents, including rats and mice; bovines; equines; ovines; felines; and canines. “Mammal” means a member or members of any mammalian species. Non-human animal models, i.e., mammals, non-human primates, murines, lagomorpha, etc. may be used for experimental investigations. The term “patient” refers to a human subject.

The term “modulator” refers to a compound or composition that modulates the level of a target, or the activity or function of a target, which may be, but is not limited to, apelin receptor. In some embodiments, the modulator compound can agonize or activate the target, such as apelin receptor. An agonist or activator of a target can increase the level of activity or signaling associated with the target.

The terms “treating,” “treatment,” and grammatical variations thereof are used in the broadest sense understood in the clinical arts. Accordingly, the terms do not require cure or complete remission of disease, and the terms encompass obtaining any clinically desired pharmacologic and/or physiologic effect, including improvement in physiologic measures associated with “normal”, non-pathologic, aging. Unless otherwise specified, “treating” and “treatment” do not encompass prophylaxis.

The phrase “therapeutically effective amount” refers to the amount of a compound that, when administered to a mammal or other subject for treating a disease, condition, or disorder, is sufficient to effect treatment of the disease, condition, or disorder. The “therapeutically effective amount” may vary depending on the compound, the disease and its severity and the age, weight, etc., of the subject to be treated.

Ranges: throughout this disclosure, various aspects of the disclosure are presented in a range format. Ranges include the recited endpoints. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.1% from the stated value. Where a percentage is provided with respect to an amount of a component or material in a composition, the percentage should be understood to be a percentage based on weight, unless otherwise stated or understood from the context.

It should be understood that the order of steps or order for performing certain actions is immaterial so long as the present disclosure remain operable. Moreover, two or more steps or actions can be conducted simultaneously.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that is generally safe, non-toxic and neither biologically nor otherwise undesirable, and include an excipient, diluent, carrier, and adjuvant that is acceptable for veterinary use as well as human pharmaceutical use. The phrase “pharmaceutically acceptable excipient” includes both one and more than one such excipient, diluent, carrier, and/or adjuvant.

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 10 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group and an alkyl group having 1 to 4 carbon atoms may be referred to as a (C₁-C₄)alkyl. This nomenclature may also be used for alkyl groups with differing numbers of carbon atoms. The term “alkyl may also be used when an alkyl group is a substituent that is further substituted in which case a bond between a second hydrogen atom and a C atom of the alkyl substituent is replaced with a bond to another atom such as, but not limited to, a halogen, or an O, N, or S atom. For example, a group —O—(C₁-C₆ alkyl)-OH will be recognized as a group where an —O atom is bonded to a C₁-C₆ alkyl group and one of the H atoms bonded to a C atom of the C₁-C₆ alkyl group is replaced with a bond to the O atom of an —OH group. As another example, a group —O—(C₁-C₆ alkyl)-O—(C₁-C₆ alkyl) will be recognized as a group where an —O atom is bonded to a first C₁-C₆ alkyl group and one of the H atoms bonded to a C atom of the first C₁-C₆ alkyl group is replaced with a bond to a second O atom that is bonded to a second C₁-C₆ alkyl group.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E-form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂-C₆)alkenyl group.

“Alkynyl” refers to an unsaturated branched or straight-chain hydrocarbon having at least one carbon-carbon triple bond derived by the removal of one hydrogen atom from a single carbon atom of a parent alkyne. Typical alkynyl groups include, but are not limited to, ethynyl; propynyl; butynyl, 2-pentynyl, 3-pentynyl, 2-hexynyl, 3-hexynyl and the like. In certain embodiments, an alkynyl group has 2 to 20 carbon atoms and in other embodiments, has 2 to 6 carbon atoms. An alkynyl group having 2 to 6 carbon atoms may be referred to as a —(C₂-C₆)alkynyl group.

“Alkoxy” refers to a radical —OR where R represents an alkyl group as defined herein. Representative examples include, but are not limited to, methoxy, ethoxy, propoxy, butoxy, cyclohexyloxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as —O—(C₁-C₆) alkyl or as —O—(C₁-C₆ alkyl) groups. In some embodiments, an alkoxy group may include 1 to 4 carbon atoms and may be designated as —O—(C₁-C₄) alkyl or as —O—(C₁-C₄ alkyl) groups group.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₆-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein.

“Carbonyl” refers to the radical —C(O) or —C(═O) group.

“Carboxy” refers to the radical —C(O)OH.

“Cyano” refers to the radical —CN.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, cycloheptane, cyclooctane, and the like. Cycloalkyl groups may be described by the number of carbon atoms in the ring. For example a cycloalkyl group having 3 to 7 ring members may be referred to as a (C₃-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C₃-C₅)cycloalkyl, a (C₃-C₇)cycloalkyl, a (C₃-C₆)cycloalkyl, or a (C₄-C₇)cycloalkyl group and these may be referred to as C₃-C₁₀ cycloalkyl, C₃-C₈ cycloalkyl, C₃-C₇ cycloalkyl, C₃-C₆ cycloalkyl, or C₄-C₇ cycloalkyl groups using alternative language.

“Heterocyclyl” refers to a cyclic group that includes at least one saturated or unsaturated, but non-aromatic, cyclic ring. Heterocyclyl groups include at least one heteroatom as a ring member. Typical heteroatoms include O, S and N and are independently chosen. Heterocyclyl groups include monocyclic ring systems and bicyclic ring systems. Bicyclic heterocyclyl groups include at least one non-aromatic ring with at least one heteroatom ring member that may be fused to a cycloalkyl ring or may be fused to an aromatic ring where the aromatic ring may be carbocyclic or may include one or more heteroatoms. The point of attachment of a bicyclic heterocyclyl group may be at the non-aromatic cyclic ring that includes at least one heteroatom or at another ring of the heterocyclyl group. For example, a heterocyclyl group derived by removal of a hydrogen atom from one of the 9 membered heterocyclic compounds shown below may be attached to the rest of the molecule at the 5-membered ring or at the 6-membered ring.

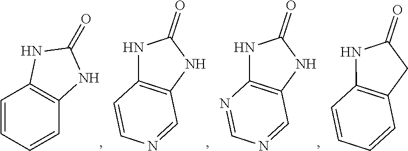

In some embodiments, a heterocyclyl group includes 5 to 10 ring members of which 1, 2, 3 or 4 or 1, 2, or 3 are heteroatoms independently selected from O, S, or N. In other embodiments, a heterocyclyl group includes 3 to 7 ring members of which 1, 2, or 3 heteroatoms are independently selected from O, S, or N. In such 3-7 membered heterocyclyl groups, only 1 of the ring atoms is a heteroatom when the ring includes only 3 members and includes 1 or 2 heteroatoms when the ring includes 4 members. In some embodiments, a heterocyclyl group includes 3 or 4 ring members of which 1 is a heteroatom selected from O, S, or N. In other embodiments, a heterocyclyl group includes 5 to 7 ring members of which 1, 2, or 3 are heteroatoms independently selected from O, S, or N. Typical heterocyclyl groups include, but are not limited to, groups derived from epoxides, aziridine, azetidine, imidazolidine, morpholine, piperazine, piperidine, hexahydropyrimidine, 1,4,5,6-tetrahydropyrimidine, pyrazolidine, pyrrolidine, quinuclidine, tetrahydrofuran, tetrahydropyran, benzimidazolone, pyridinone, and the like. Substituted heterocyclyl also includes ring systems substituted with one or more oxo (═O) or oxide (—O⁻) substituents, such as piperidinyl N-oxide, morpholinyl-N-oxide, 1-oxo-1-thiomorpholinyl, pyridinonyl, benzimidazolonyl, benzo[d]oxazol-2(3H)-only, 3,4-dihydroisoquinolin-1(2H)-only, indolin-only, 1H-imidazo[4,5-c]pyridin-2(3H)-only, 7H-purin-8(9H)-only, imidazolidin-2-only, 1H-imidazol-2(3H)-only, 1,1-dioxo-1-thiomorpholinyl, and the like.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, an alkyl group in which each of the hydrogen atoms is replaced with a halogen atom. For example, the term “perhaloalkyl”, includes, but is not limited to, trifluoromethyl, pentachloroethyl, 1,1,1-trifluoro-2-bromo-2-chloroethyl, and the like.

“Heteroaryl” refers to a monovalent heteroaromatic group derived by the removal of one hydrogen atom from a single atom of a parent heteroaromatic ring system. Heteroaryl groups typically include 5- to 14-membered, but more typically include 5- to 10-membered aromatic, monocyclic, bicyclic, and tricyclic rings containing one or more, for example, 1, 2, 3, or 4, or in certain embodiments, 1, 2, or 3, heteroatoms chosen from O, S, or N, with the remaining ring atoms being carbon. In monocyclic heteroaryl groups, the single ring is aromatic and includes at least one heteroatom. In some embodiments, a monocyclic heteroaryl group may include 5 or 6 ring members and may include 1, 2, 3, or 4 heteroatoms, 1, 2, or 3 heteroatoms, 1 or 2 heteroatoms, or 1 heteroatom where the heteroatom(s) are independently selected from 0, S, or N. In bicyclic aromatic rings, both rings are aromatic. In bicyclic heteroaryl groups, at least one of the rings must include a heteroatom, but it is not necessary that both rings include a heteroatom although it is permitted for them to do so. For example, the term “heteroaryl” includes a 5- to 7-membered heteroaromatic ring fused to a carbocyclic aromatic ring or fused to another heteroaromatic ring. In tricyclic aromatic rings, all three of the rings are aromatic and at least one of the rings includes at least one heteroatom. For fused, bicyclic and tricyclic heteroaryl ring systems where only one of the rings contains one or more heteroatoms, the point of attachment may be at the ring including at least one heteroatom or at a carbocyclic ring. When the total number of S and O atoms in the heteroaryl group exceeds 1, those heteroatoms are not adjacent to one another. In certain embodiments, the total number of S and O atoms in the heteroaryl group is not more than 2 In certain embodiments, the total number of S and O atoms in the aromatic heterocycle is not more than 1 Heteroaryl does not encompass or overlap with aryl as defined above. Examples of heteroaryl groups include, but are not limited to, groups derived from acridine, carbazole, cinnoline, furan, imidazole, indazole, indole, indolizine, isobenzofuran, isochromene, isoindole, isoquinoline, isothiazole, 2H-benzo[d][1,2,3]triazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and the like. In certain embodiments, the heteroaryl group can be between 5 to 20 membered heteroaryl, such as, for example, a 5 to 14 membered or 5 to 10 membered heteroaryl. In certain embodiments, heteroaryl groups can be those derived from thiophene, pyrrole, benzothiophene, 2H-benzo[d][1,2,3]triazole benzofuran, indole, pyridine, quinoline, imidazole, benzimidazole, oxazole, tetrazole, and pyrazine.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

5.16. Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-52 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
Aspect 1. A method of reducing blood-brain barrier (BBB) permeability in a subject in need thereof, comprising administering to the subject a therapeutically effective amount of an apelin receptor agonist to reduce BBB permeability.
Aspect 2. A method of treating a disorder related to increased BBB permeability in a subject, comprising administering to a subject in need thereof a therapeutically effective amount of an apelin receptor agonist.
Aspect 3. A method of treating neurodegeneration or a neurodegenerative disease in a subject, comprising administering to a subject in need thereof a therapeutically effective amount of an apelin receptor agonist.
Aspect 4. A method of reducing neuro-inflammation in a subject, comprising administering to a subject in need thereof a therapeutically effective amount of an apelin receptor agonist.
Aspect 5. A method of treating a neurodegenerative disease in a subject, comprising administering to a subject in need thereof a therapeutically effective amount of an apelin receptor agonist.
Aspect 6. The method of any one of any one of aspects 1-6, wherein the subject exhibits cognitive impairment.
Aspect 7. The method of any one of aspects 1-7, wherein the subject has age-related cognitive impairment.
Aspect 8. The method of any one of aspects 1-8, wherein the subject has dementia (e.g. acute, chronic, or progressive dementia).
Aspect 9. The method of any one of aspects 1-9, wherein the subject has neurodegeneration.
Aspect 10. The method of any one of aspects 1-9, wherein the subject has cognitive impairment.
Aspect 11. The method of any one of aspects 1 to 10, wherein the subject has acute cognitive impairment (e.g., cognitive impairment associated with acute inflammation).
Aspect 12. The method of any one of aspects 1 to 11, wherein the subject has postoperative cognitive dysfunction (POCD).
Aspect 13. The method of any one of aspects 1 to 12, wherein the subject has traumatic brain injury (TBI).
Aspect 14. The method of any one of aspects 1 to 13, wherein the subject has intensive care unit (ICU) delirium, post-operative delirium, delirium due to trauma, or delirium due to bone fracture (e.g., hip fracture).
Aspect 15. The method of aspects 14, wherein post-operative delirium is following cardiovascular surgery.
Aspect 16. The method of any one of aspects 1 to 15, wherein the subject is on a ventilator.
Aspect 17. The method of any one of aspects 1 to 16, wherein the subject has neuroinflammation (e.g., such as peripheral inflammation).
Aspect 18. The method of any one of aspects 1-4, wherein the subject is in need of treatment of a neurodegenerative disease.
Aspect 19. The method of any one of aspects 1 to 17, wherein the subject has a neurodegenerative disease selected from Alzheimer's disease (AD), vascular dementia (VaD), Parkinson's disease (PD), amyotrophic lateral sclerosis (ALS), stroke, Huntington's disease (HD), and multiple sclerosis (MS).
Aspect 20. The method of any one of aspects 1-3, 4, and 6-17, wherein the subject does not have a neurodegenerative disease,
Aspect 21. The method of any one of aspects 1 to 20 wherein the subject is human and at least 40-years-old.
Aspect 22. The method of aspect 21, wherein the subject is at least 50-years-old.
Aspect 23. The method of aspect 22, wherein the subject is at least 60-years-old.
Aspect 24. The method of aspect 23, wherein the subject is at least 65-years-old.
Aspect 25. The method of aspect 24, wherein the subject is at least 70-years-old.
Aspect 26. The method of aspect 25, wherein the subject is at least 75-years-old.
Aspect 27. The method of aspect 26, wherein the subject is at least 80-years-old.
Aspect 28. The method of any of aspects 1 to 27, wherein the subject has, or is identified as having, a low circulating level of apelin.
Aspect 29. The method of any one of aspects 1 to 28, wherein the apelin receptor agonist is of formula (I) or (II):

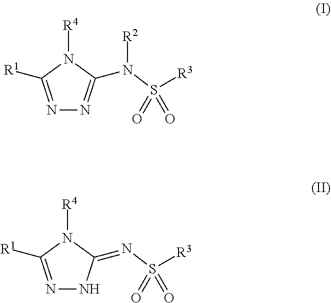

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and further wherein:
if R⁴ is an unsubstituted or substituted phenyl ring and R³ is a group of formula (CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.
Aspect 30. The method of aspect 29, wherein R¹ is an unsubstituted pyridyl or is a pyridyl substituted with 1 or 2 R^{1a} substituents.
Aspect 31. The method of aspect 29 or 30, wherein Rain each instance is independently selected from —CH₃, —CH₂CH₃, —F, —Cl, —Br, —CN, —CF₃, —CH═CH₂, —C(═O)NH₂, —C(═O)NH(CH₃), —C(═O)N(CH₃)₂, —C(═O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula

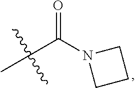 [SMILES: *C(=O)N1CCC1]

wherein the symbol [symbol] , when drawn across a bond, indicates the point of attachment to the rest of the molecule.
Aspect 32. The method of any one of aspects 29 to 31, wherein R¹ is selected from

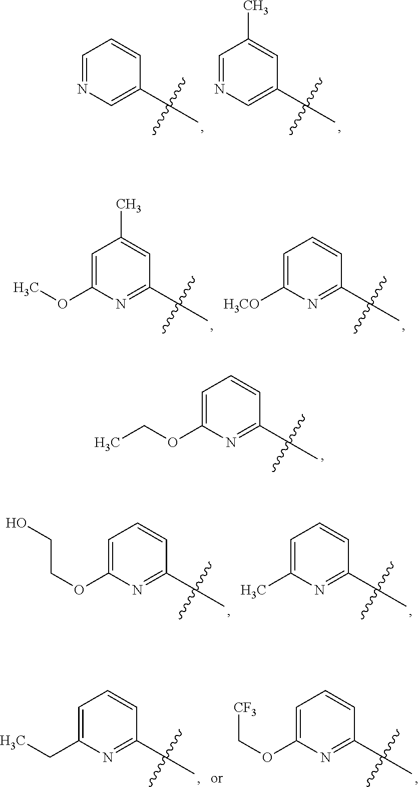

wherein the symbol [symbol] when drawn across a bond, indicates the point of attachment to the rest of the molecule.
Aspect 33. The method of any one of aspects 29 to 32, wherein R² is —H.
Aspect 34. The method of any one of aspects 29 to 33, wherein R⁴ is a phenyl, pyridyl, pyrimidinyl, isoxazolyl, indolyl, naphthyl, or pyridinyl any of which may be unsubstituted or substituted with 1, 2, or 3 R^{4a} substituents.
Aspect 35. The method of aspect 34, wherein R⁴ is a phenyl substituted with 1 or 2 R^{4a} substituents.
Aspect 36. The method of aspect 35, wherein the 1 or 2 R^{4a} substituents are —O—(C₁-C₂ alkyl) groups.
Aspect 37. The method of any one of aspects 29 to 36, wherein R^{4a} is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.
Aspect 38. The method of any one of aspects 29 to 37, wherein R³ is selected from a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula (CR^{3b}═CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.
Aspect 39. The method of any one of aspects 29 to 38, wherein Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyrazolyl, or oxetanyl any of which may be unsubstituted or substituted with 1, 2, or 3, R^{Q} substituents.
Aspect 40. The method of any one of aspects 29 to 39, wherein Q is a monocyclic heteroaryl group with 5 or 6 ring members containing 1 or 2 heteroatoms selected from N, O, or S and Q is unsubstituted or is substituted with 1 or 2 R^{Q} substituents.
Aspect 41. The method of any one of aspects 29 to 40, wherein R³ is a group of formula —(CR^{3d}R^{3e})—(CR^{3f}R^{3g})-Q.
Aspect 42. The method of any one of aspects 29 to 41, wherein R³ has the formula

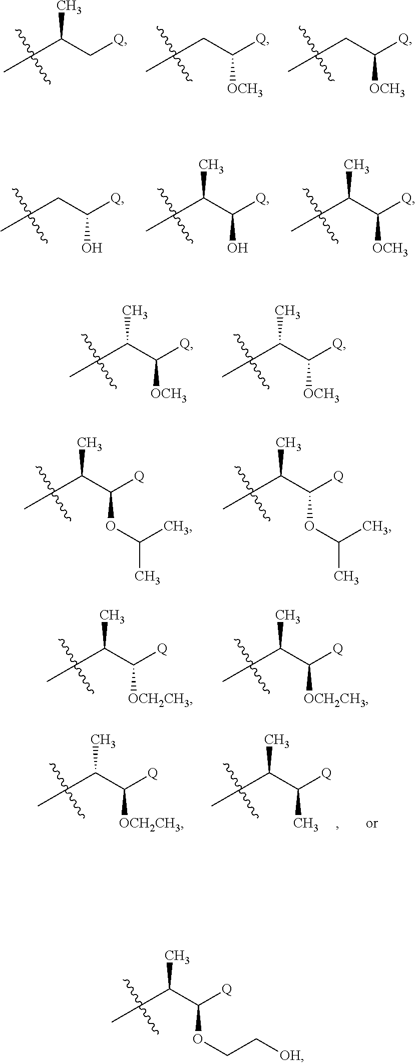

wherein the symbol [symbol] , when drawn across a bond, indicates the point of attachment to the rest of the molecule.
Aspect 43. The method of any one of aspects 29 to 42, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.
Aspect 44. The method of aspect 43, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide or a pharmaceutically acceptable salt thereof.
Aspect 45. The method of any one of aspects 1 to 44, wherein the apelin receptor agonist is administered intravenously or intrathecally.
Aspect 46. The method of any one of aspects 1 to 45, wherein the apelin receptor agonist is administered orally.
Aspect 47. The method of any one of aspects 1 to 46, wherein the dose is administered daily.
Aspect 48. The method of any one of aspects 1 to 47, wherein the dose is administered as a plurality of equally or unequally divided sub-doses.
Aspect 49. The method of any one of aspects 1 to 48, wherein the dose is administered at varying dosing intervals.
Aspect 50. The method of any one of aspects 1 to 49, wherein the dose is 200 mg.
Aspect 51. The method of any one of aspects 1 to 50, further comprising, assessing cognitive function after the dosing.
Aspect 52. The method of aspect 51, wherein the cognitive function is assessed at least one day after dosing (e.g., at least one week, or at least one month after dosing).

### 6. EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature

6.1. Example 1: Bioinformatic Analyses Identify Relationships Between Apelin and Cognitive Decline in Human Healthy Aging Cohorts

A survival predictor model was used to examine the relationship between serum levels of apelin and future risk of cognitive decline in human healthy aging cohorts, using unpublished clinical outcome data and proteomics data generated on archived samples, based on survival modeling. A Cox proportional hazards model was used, with a hazard ratio and associated p-value generated for apelin.

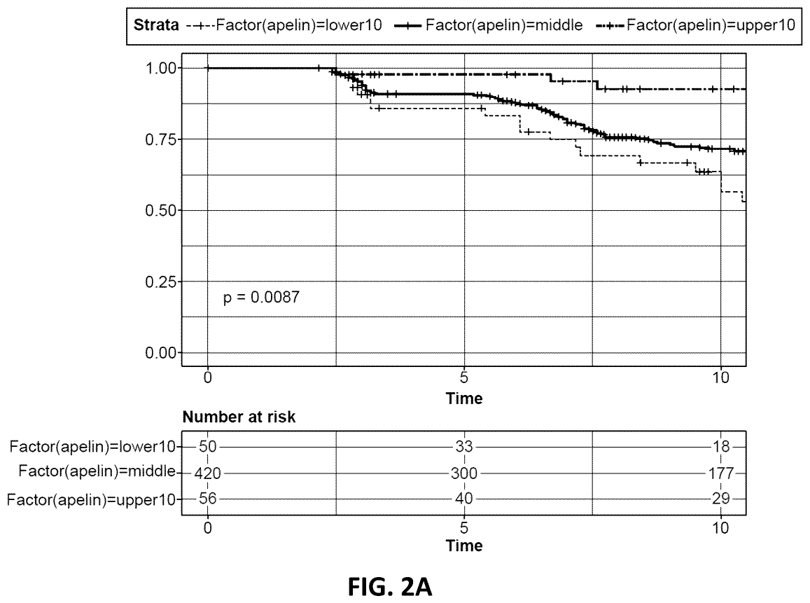

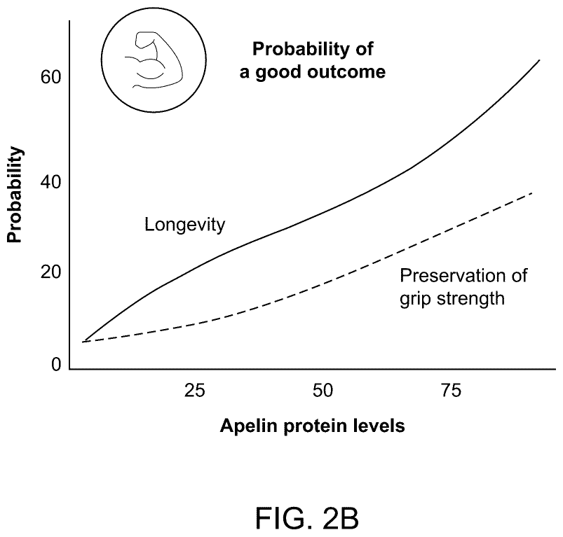

As shown in FIGS. 2A-2B, the human aging platform presented by the inventors revealed protective associations between higher circulating apelin protein levels and healthspan outcomes. The graph showing probability of a good outcome shows that serum apelin concentration is associated with a higher probability of preservation of grip strength and longevity across decades of follow-up. The graph showing preservation of cognitive function shows that the upper and lower 10th percentiles of serum apelin concentrations are associated with a preservation of cognitive function (as assessed by CASI score decline) across 10 years of follow-up. A Hazard ratio of 0.85 was determined for cognitive decline based on cognitive abilities screening instrument (CASI) score (p=0.0292). Based on this result, an exemplary apelin receptor agonist was assessed in in vivo in models of BBB permeability and neurodegenerative disease.

6.1.1. Analysis

Proteomics data was used to calculate the change (delta) in apelin levels between exam 3 and exam 4 in the Hawaiian cohort. It was found that the change in apelin levels (deltaAPLN) is associated with higher probability of CASI decline, shown by survival analysis (COX proportional hazard model). The COXPH model was adjusted for exam 3 age (Vagex3), smoking status (pack years-PACKYRX3), alcohol intake (ALCX3), and CASI score at exam 4 (CASIX4). The coefficients from the model are shown below, where the second column (exp(coef)) represents the Hazard Ratio, and the right most column shows the corresponding p-value.

| | coef | exp (coef) | se (coef) | z Pr(>\|z \|) | | |
|---|---|---|---|---|---|---|
| Vagex3 | 0.1155917 | 1.1225375 | 0.0313001 | 3.693 | 0.000222 | *** |
| PACKYRX3 | 0.0005354 | 1.0005355 | 0.0032928 | 0.163 | 0.870842 | |
| ALCX3 | −0.0006474 | 0.9993528 | 0.0033086 | −0.196 | 0.844856 | |
| CASIX4 | −0.1547559 | 0.8566243 | 0.0136341 | −11.351 | < 2e−16 | *** |
| deltaAPLN | −0.1458925 | 0.8642506 | 0.0738962 | −1.974 | 0.048349 | * |

FIGS. 2A-2B show the Kaplan Meir curve of the bioinformatic analysis. The Y-axis of FIGS. 2A-2B represent the probability for CASI decline. The X axis of FIGS. 2A-2B represent time in years (10 yrs). The graph represents cohort participants whose APLN level was in the lower 10% of the cohort (Factor (apelin) lower), and represents the participants with the highest APLN levels (Factor (apelin) upper) (upper 10%). The middle line (Factor (apelin) middle) is between 10-90%.

6.2. Example 2: BGE-105 Rescues BBB Breakdown in LPS Induced BBB Permeability

The activity of BGE-105 on BBB permeability was assessed in 12-month-old mice challenged with lipopolysaccharide (LPS) to induce an increase in BBB permeability. Lipopolysaccharide (LPS) induces inflammation and BBB disruption in mice.

6.2.1. Methods

Mice were handled in accordance with principles and guidelines established by the “Guide for the Care and Use of Laboratory Animals” and overseen by the BioAge Lab's institutional animal care and use committee (IACUC). Mice were divided into three experimental Groups:
Group 1: (Vehicle Control). Mice administered a vehicle via P.O.
Group 2: (Vehicle+LPS). Mice were administered a vehicle via P.O. for 1 week followed by LPS on day 8, and
Group 3: (Treatment with BGE-105). Mice were administered with BGE-105 via P.O. for 1 week followed by LPS on day 8.

Note that the vehicle solution for LPS is sterile, normal saline. The vehicle solution for BGE-105 is 2% HPMC and 1% Pluronic F68. for a schematic of the experimental protocol is shown in FIG. 3A. Mice in Group 3 were administered BGE-105 at 50 mg/kg bis in die (BID) for one week (7 days) before being exposed to the LPS challenge on day 8. The healthy control group mice received P.O. vehicle (BID) for one week (7 days), followed by one intraperitoneal injection of normal saline. P.O. vehicle BID or BGE-105 BID were kept until the endpoint Evans blue assay to measure BBB permeability. BID represents twice-daily dosing of either vehicle or BGE-105.

23 hours after LPS or normal saline injection, mice were intravenously injected Evans Blue (EB) (2%, 75 ul/30 g BW). One hour later, blood samples were collected via retro-orbital bleeding under isoflurane anesthesia. Mice were euthanized via transcardiac perfusion under isoflurane anesthesia before the whole brain was resected to allow for the microdissection of the specific brain regions (olfactory bulb, hippocampus) from each hemisphere. Brain sections were massed, flash frozen in liquid nitrogen and stored at −80° C. until further use.

Frozen olfactory bulb (OB) and hippocampus (HPF) sections were placed in 200 μl of extraction buffer (50% trichloroacetic acid in formamide) for 24 hours. Following mechanical homogenization, samples were spun at 12,000 g for 30 min at 4° C. 30 μl of each sample was added to a microplate in duplicate, along with an Evans blue standard dilution series, and all wells were diluted with 90111 of 95% ethanol to ensure optical path consistency. Endpoint fluorescence was recorded (620 nm excitation, 680 nm emission) with a SpectraMax iD5 (Molecular Devices), normalized to tissue weight, and relative LPS-related change in fluorescence (a measure of blood-brain barrier leakage) was calculated compared to vehicle- or BGE-105 treated mice.

6.2.2. Results

As shown in FIG. 3B, the LPS challenge group (Group 2) increased BBB permeability in the OB (+17%) and in the HPF (+39%; p<0.01, one-way ANOVA with Tukey's multiple comparisons) compared to the healthy control Group 1 (control versus LPS). Pretreatment of BGE-105 (Group 3) significantly reversed the LPS-induced increase in BBB permeability in the OB and the HPF of the treated mice (p<0.05, one-way ANOVA with Tukey's multiple comparisons) (LPS versus LPS+BGE-105).

The activity of exemplary Compound 3 (BGE-105; e.g., as described herein) was assessed according to the methods described above.

6.3. Example 3: BGE-105 Rescues BBB Breakdown of BBB Permeability in Naturally Aged Mice

Next, the activity of BGE-105 on BBB permeability was assessed in naturally aged mice exhibiting age-related increase in BBB permeability.

6.3.1. Methods

The mice were handled in accordance with principles and guidelines established by the “Guide for the Care and Use of Laboratory Animals” and overseen by the BioAge Lab's institutional animal care and use committee (IACUC). Mice were housed conventionally in a constant temperature (20-22° C.) and humidity (40%-60%) animal room with a 12/12 h light/dark cycle and free access to food and water.

FIG. 4A illustrates the establishment of an aged mouse model for age-related increase in BBB permeability. As shown in FIG. 4A, in aged mice (22-month old mice) BBB permeability is increased in both the olfactory bulb and hippocampus regions of the brain, as compared to young (3-month-old) or adult (13 month old) mice, as measured using Evans blue (EB) staining assay (e.g., as described herein).

For assessment of BGE-105 activity, the age-dependent BBB permeability model was established using 26-month-old female C57BL/6 mice. Mice were randomized based on the body weight and body condition scores to two groups (vehicle or BGE-105, n=8). Mice were administered P.O. vehicle (Control) or BGE-105 via P.O. at 50 mg/kg (BID) for one week (7 days). See FIG. 4B for a schematic of the protocol. The BBB permeability was measured via Evans blue assay. Mice were intravenously injected with Evans Blue (2%, 75 ul/30 g BW). One hour later, blood samples were collected via retro-orbital bleeding under isoflurane anesthesia. Mice were euthanized via transcardiac perfusion under isoflurane anesthesia before the whole brain was resected to allow for the microdissection of the specific brain regions (olfactory bulb, hippocampus) from each hemisphere. Brain sections were massed, flash frozen in liquid nitrogen and stored at −80° C. until further use.

Frozen olfactory bulb and hippocampus were placed in 200 μl of extraction buffer (50% trichloroacetic acid in formamide) for 24 hours. Following mechanical homogenization, samples were spun at 12,000 g for 30 min at 4° C. 30 μl of each sample was added to a microplate in duplicate, along with an Evans blue standard dilution series, and all wells were diluted with 90111 of 95% ethanol to ensure optical path consistency. Endpoint fluorescence was recorded (620 nm excitation, 680 nm emission) with a SpectraMax iD5 (Molecular Devices), normalized to tissue weight, and relative treatment-related change in fluorescence (a measure of blood-brain barrier leakage) was calculated compared to untreated aged mice.

6.3.2. Results

As shown in FIG. 4C, pretreatment of BGE-105 significantly reversed the age-induced increase in BBB permeability in the OB (−28%; p<0.01, Mann-Whitney U-test) (aged versus aged+BGE-105). The activity of exemplary Compound 3 (BGE-105; e.g., as described herein) was assessed according to the methods described above. Based on these results, it was found that BGE-105 restores BBB integrity in aged mice.

6.4. Example 4: BGE-105 Efficacy in Age-Dependent Peripheral Inflammation

Next, the activity of BGE-105 on age-dependent peripheral inflammation was assessed in aged mice, with the hypothesis that age-related neuroinflammation plays a role in age-related BBB permeability and neurodegeneration.

### Methods

The mice were handled in accordance with principles and guidelines established by the “Guide for the Care and Use of Laboratory Animals” and overseen by the BioAge Lab's institutional animal care and use committee (IACUC). Mice were housed conventionally in a constant temperature (20-22° C.) and humidity (40%-60%) animal room with a 12/12 h light/dark cycle and free access to food and water. 26 month-old mice were randomized based on the body weight and body condition scores to two groups (vehicle or BGE-105, n=8). 4 month-old female mice were used as an additional control group (vehicle, n=4). Mice were administered P.O. vehicle or BGE-105 at 50 mg/kg (BID) for one week. After completion of dosing, blood samples were collected via retro-orbital bleeding under isoflurane anesthesia into K2-EDTA tubes to prevent coagulation. Mice were euthanized via transcardiac perfusion under isoflurane anesthesia. Whole blood was centrifuged to isolate plasma, which was stored at −80° C. until further experimentation.

For qualitative analysis of 40 different cytokines, plasma samples were assessed with the Mouse Cytokine Array Panel (R&D Systems) according to manufacturer's instructions. The array was developed using chemiluminescent detection and imaged using the ChemiDoc XRS+ system (BioRad). The optical of each dot on the array serves as a relative measure of cytokine abundance (e.g., a more intensely-stained dot denotes more cytokine present in the sample) and was analyzed with ImageJ software (NIH).

For quantitative analysis, plasma samples were assessed with Quantikine® enzyme-linked immunosorbent assays (ELISAs) for mouse CXCL1 and CXCL13 (R&D Systems) according to manufacturer's instructions. Optical density of the completed ELISA was imaged using a SpectraMax iD5 (Molecular Devices), sample concentrations were calculated compared to a standard curve, and relative change with age+/−BGE-105 treatment was calculated with young mouse plasma serving as the normalization.

### Result:

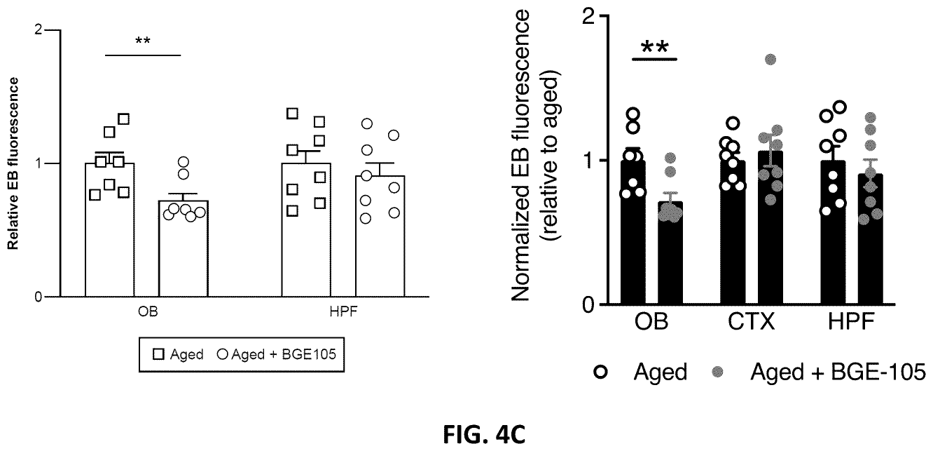

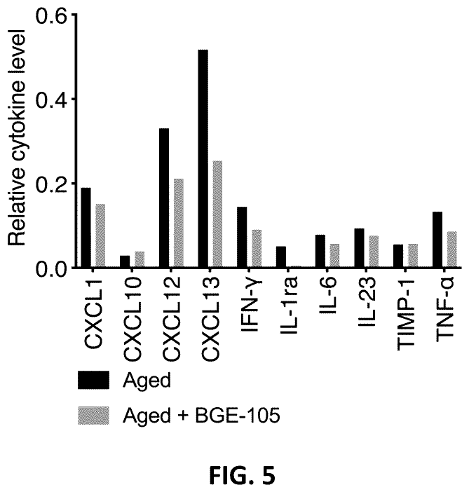

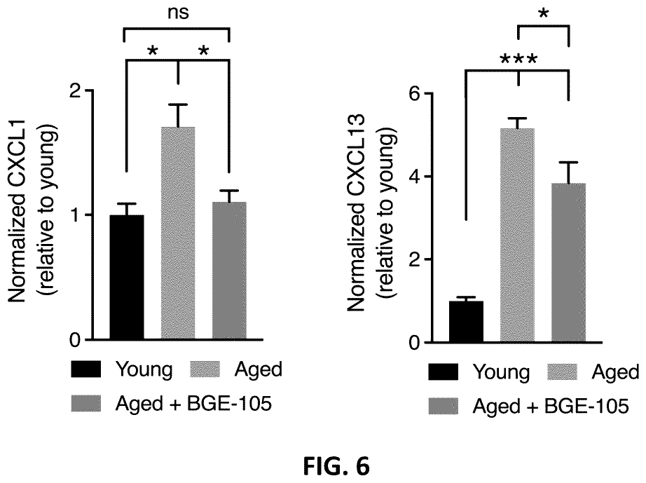

In the cytokine array, 10 (CXCL1, CXCL10, CXCL12, CXCL13, IFN-γ, IL-1Rα, IL-6, IL-23, TIMP-1, TNF-α) out of 40 (25%) of all cytokines assessed were detectable. Within these cytokines, treatment with BGE-105 decreased the optical density by an average of 28% (FIG. 5). In the ELISAs, CXCL1 concentrations in plasma were reduced in aged mice with BGE-105 treatment by 35% (one-way ANOVA with Tukey's multiple comparisons test; *, p<0.05) (FIG. 6). As shown in FIG. 6, CXCL13 concentrations in plasma were reduced in aged mice with BGE-105 treatment by 26% (one-way ANOVA with Tukey's multiple comparisons test; *, p<0.05) (FIG. 6).

The results of FIGS. 5 and 6 show that treatment with BGE-105 decreases peripheral inflammation.

The results of FIGS. 5 and 6 show that treatment with BGE-105 showed that BGE-105 decreased circulating levels of two cytokines (CXCL1/13) associated with mortality, neutrophil recruitment, and propagation of inflammation.

6.5. Example 5: BGE-105 Efficacy in Age-Dependent Hippocampal Brain-Derived Neurotrophic Factor (BDNF) Expression

Next, the activity of BGE-105 on age-dependent hippocampal BDNF expression was assessed in aged mice. BDNF belongs to a family of neurotrophins. BDNF plays an important role in neuronal survival and growth, serves as a neurotransmitter modulator, and participates in neuronal plasticity, which is essential for learning and memory, associated with cognitive function.

### Methods:

The mice were handled in accordance with principles and guidelines established by the “Guide for the Care and Use of Laboratory Animals” and overseen by the BioAge Lab's institutional animal care and use committee (IACUC). Mice were housed conventionally in a constant temperature (20-22° C.) and humidity (40%-60%) animal room with a 12/12 h light/dark cycle and free access to food and water. 26 month-old mice were randomized based on the body weight and body condition scores to two groups (vehicle or BGE-105, n=8). Mice were administered P.O. vehicle or BGE-105 at 50 mg/kg (BID) for one week. After completion of dosing, mice were euthanized via transcardiac perfusion under isoflurane anesthesia, the whole brain was resected, and the entirety of the hippocampus was extracted, massed, and flash frozen in liquid nitrogen before being stored at −80° C.

Hippocampal samples were homogenized with 30 volumes of radioimmunoprecipitation immunoassay (RIPA) buffer with Halt Protease and Phosphatase Inhibitor (Thermofisher) before being spun at 16,000×g for 20 minutes at 4° C. and supernatant removed to new, labeled tube. Total protein concentrations for each sample were analyzed with Pierce BCA Protein Assay Kit (Thermofisher) according to manufacturer's instructions, then all samples were diluted to 0.2 μg/μL with RIPA buffer.

For quantitative analysis of hippocampal BDNF expression, tissue samples were assessed with Quantikine® enzyme-linked immunosorbent assay (ELISA) for Total BDNF (R&D Systems) according to manufacturer's instructions. Optical density of the completed ELISA was imaged using a SpectraMax iD5 (Molecular Devices), sample concentrations were calculated compared to a standard curve, and relative change with age+/−BGE-105 treatment was calculated with young mouse plasma serving as the normalization.

### Result:

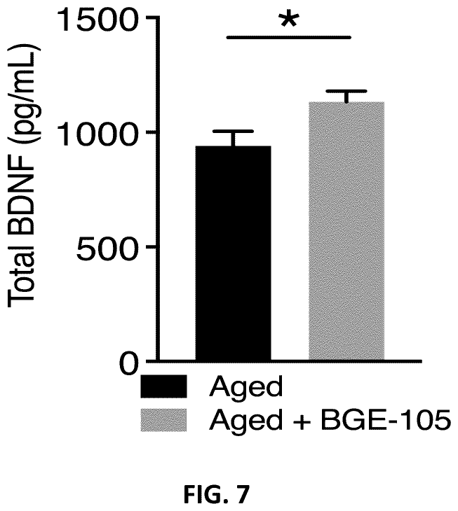

As shown in FIG. 7, in the BDNF ELISA, which measures both free BDNF and BDNF bound to its receptor TrkB, oral treatment with BGE-105 increased the concentration of total BDNF in the hippocampus in aged mice by 20% (unpaired two-sided t-test; *,p<0.05).

The results show that oral administration of BGE-105 increased the production/release of a pro-neuronal survival factor, BDNF, which typically declines with age and neurodegeneration.

6.6. Example 6: Effects of BGE-105 on Neuroinflammation

Neuroinflammation is associated with neurodegenerative diseases, such as Alzheimer's disease, Parkinson's disease, and amyotrophic lateral sclerosis (ALS). Astrocytes are known to express Apelin receptor (Apinr) and are the principal cell type that express APJ in the CNS. Astrocytes undergo an inflammatory transition after infections, acute injuries, and chronic neurodegenerative diseases. Astrocytes are critical components of the neurovascular unit that support blood-brain barrier (BBB) function. Pathological transformation of astrocytes to reactive states can be protective or harmful to BBB function.

The activity of BGE-105 was assessed to understand BGE-105 and its activity at the apelin receptor in mitigating the effects of inflammatory stimuli in cultured brain-specific immune cells, extending the potential action of BGE-105 to brain-specific cells.

In vitro experiments were performed in mouse or human cells (fresh, primary cells or transformed, stable cell lines) of astrocytes and microglia to explore the effects of BGE-105 on inflammation.

Reactive Astrocyte Cocktail (RAC) drives neurotoxic astrocyte reactivity as seen in aging and diseased brains. Degenerative or aging astrocytes can be recapitulated by RAC in vitro. Therefore, a RAC-induced astrocytes were used as a model of neurotoxicity. Table 1 lists common factors in a RAC.

TABLE 1
Reactive Astrocyte Cocktail (RAC)

| Factor | Concentration |
|---|---|
| TNF-α | 30 ng/ml |
| IL-1α | 3 ng/ml |
| Complement C1q | 400 ng/ml |

To demonstrate the effect of BGE-105 on astrocytes, control astrocytes or RAC-induced neurotoxic astrocytes were treated with one of the following test compounds: (a) BGE-105, an agonist of the apelin receptor, (b) Pyr⁽¹⁾-Apelin-13, a ligand of the apelin receptor, or (c) BAY-11-7082, an NF-κB inhibitor.

### Methods and Materials

Mouse astrocyte cells were seeded on 24-well plates at a low density of approximately 20 k astrocytes per well.

Stimulated Reactive Astrocyte.

To stimulate astrocytes into a reactive state found in aging and neurodegenerative diseases per previous publications (Liddelow et al., 2017; Barbar et al., 2020), a combination of the cytokines TNF-α and IL-1α, and complement factor C1q, were added at 30 ng/mL, 3 ng/mL, and 400 ng/mL, respectively, into astrocyte culture media to make ‘reactive astrocyte cocktail’ (RAC) media. Astrocytes were cultured in RAC media for 24 hours, with or without various concentrations of BGE-105 (10 nM, 50 nM, 250 nM final concentration), (Pyr¹)-Apelin-13 (50 nM; Bachem, Cat. #4029110), BAY-11-7082 (50 μM; Abcam, Cat. #ab141228) or equal volume of vehicle (0.01% DMSO in PBS) (hereafter referred to as ‘RAC treatment’). Cells in Groups 3-6 were treated with RAC for 24 hours, with or without the test compounds (n=4 per group).
Group 1: Control [CON]
Group 2: CON+BGE-105 (50 nM) [CON+105]
Group 3: Reactive control treated with RAC only [RAC]
Group 4: RAC+BGE-105 (50 nM) [RAC+105]
Group 5: RAC+Pyrm-Apelin-13 (50 nM)
Group 6: RAC+BAY-11-7082 (NF-κB inhibitor; 50 μM) [RAC+BAY]

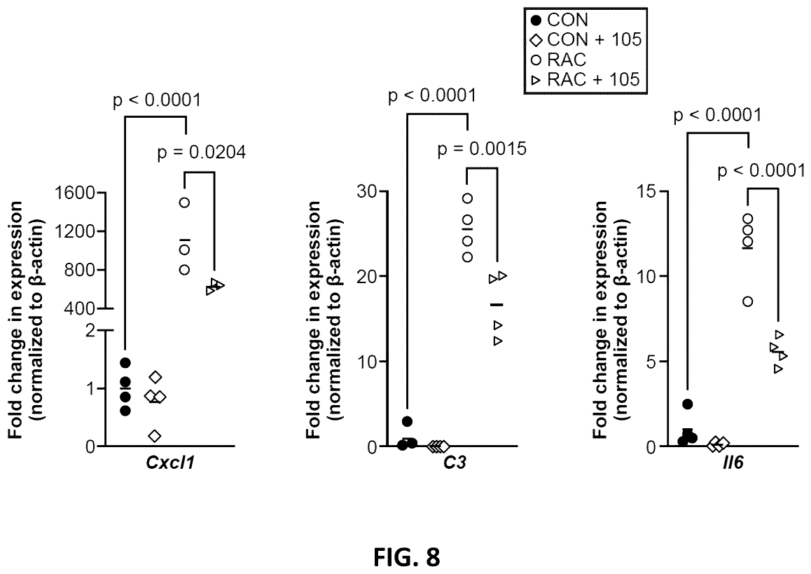

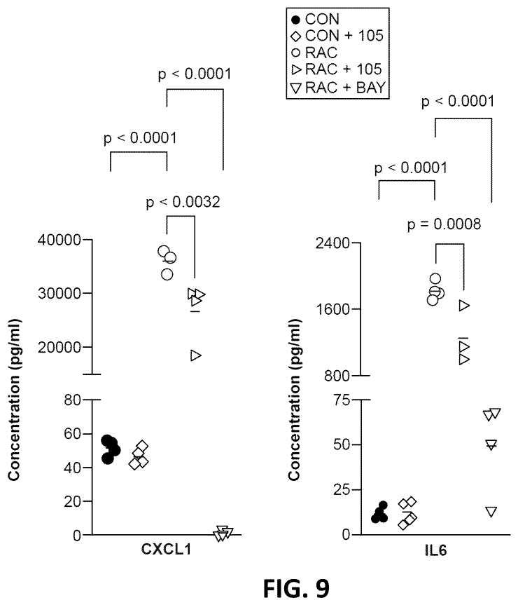

Media from each tested cell group was collected for cytokine release analysis. Cells were lysed for RNA collection and RT-PCR was performed to synthesis cDNA under standard conditions. Next, transcriptome analysis was performed to determine the expression level of astrocyte genes or astrocyte responsive genes. Changes of expression was normalized to β-actin (FIGS. 8 and 9). Exemplary biomarkers include CCL2, CXCL10, CXCL11, CXCL1, CXCL2, CXCL3, CXCL8, EDN1, SERPINA3, CIS, CTRL, C3, CFB, VEGF, and IL-6.

Mice were handled in accordance with principles and guidelines established by the “Guide for the Care and Use of Laboratory Animals” and overseen by the BioAge Lab's Institutional Animal Care and Use Committee (IACUC). Mouse primary astrocytes were isolated and cultured in 0.01% poly-L-lysine coated 6- or 12-well tissue culture plates at 200,000 or 120,000 cells/well, respectively, with astrocyte growth media as previously described in (Lundquist et al. 2022. Knockdown of Astrocytic Monocarboxylate Transporter 4 in the Motor Cortex Leads to Loss of Dendritic Spines and a Deficit in Motor Learning. Mol Neurobiol 59, 1002-1017). Embryonic rat neurons were purchased from ScienCell (Cat. #R1550) and cultured in neuronal media (ScienCell, Cat. #1521) per manufacturer's protocol, with slight modifications. Briefly, neurons were thawed and resuspended in neuronal media with the addition of 1 μg laminin (Sigma, Cat. #L2020) per 1 mL culture media, then plated in 0.01% poly-L-lysine coated 96-well tissue culture plates at 60,000 cells/well. All cells were grown in standard mammalian cell culture conditions (37° C. with 5% CO₂) and all experiments were done using two separate preparations and at least three replicates. All statistical analysis and graphs were completed in Prism (version 9; GraphPad) unless otherwise stated.

Cytokine Secretion.

For analysis of cytokines secreted by astrocytes, culture media was collected after RAC treatment, centrifuged at 10,000 RPM for 5 minutes to pellet any debris, and supernatant was transferred to a new tube and assayed immediately or frozen at −80° C.

To assess a panel of cytokines and chemokines, cell culture supernatant was diluted 1:10 and assayed using the Proteome Profiler Mouse Cytokine Array Kit, Panel A (R&D Systems, Cat. #ARY006) per manufacturer's protocol. Dot blots were analyzed in Fiji (Schindelin et al. 2012. Fiji: an open-source platform for biological-image analysis. Nat Methods 9, 676-682) and transcription factor enrichment was performed with Enrichr (Chen, et al. 2013. Enrichr: interactive and collaborative HTML5 gene list enrichment analysis tool. BMC Bioinformatics 14, 128).

Biomarker Concentration.

For individual analysis of CXCL1 or IL-6 concentrations, cell culture supernatant was diluted 1:50 or 1:10, respectively, and assessed using mouse-specific ELISA kits per manufacturer's protocols (R&D Systems, Cat. #DY453 and #DY406).

Astrocyte Protein Expression.

For analysis of astrocyte protein expression, cells were washed after RAC treatment with PBS and lysed with Neuronal Protein Extraction Reagent (Thermo Scientific, Cat. #87792), centrifuged to pellet debris, and supernatant was transferred to a new tube. Total protein concentration was measured by BCA protein assay (Thermo Scientific, Cat. #PI23227). 10 μg of protein was loaded per sample into a 10% Bis-Tris gel (Thermo Scientific, Cat. #NP0301) and run at 100V for 2 hours before a dry transfer onto a PVDF membrane (Invitrogen, Cat. #IB401031). Membranes were blocked for 1 hour at room temperature in 2% bovine serum albumin (BSA) in TBS with 0.05% Tween-20 (TBS-T) before overnight incubation at 4° C. with primary antibodies diluted as follows: rabbit anti-β-actin (1:5000, Genetex, Cat. #G109639), rabbit anti-APJ (1:500, Invitrogen, Cat. #702069), rabbit anti-phospho-AKT (Ser473; 1:1000, Cell Signaling, Cat. #4060), rabbit anti-AKT (1:1000, Cell Signaling, Cat. #4685), rabbit anti-NF-κB p65 (1:1000, Cell Signaling, Cat. #8242S), rabbit anti-phospho-IκBα (Ser32, 1:1000, Cell Signaling, Cat. #2859T), and rabbit anti-IκBα (1:1000, Cell Signaling, Cat. #4812S). Blots were washed with TBS-T and probed with goat anti-rabbit secondary antibody (1:5000, Jackson ImmunoResearch, Cat. #111-035-144) for 1 hour at room temperature in 2% BSA in TBS-T. Blots were washed with TBS-T, developed for 1 minute in enhanced chemiluminescent substrate (Thermo Scientific, Cat. #34095), and imaged on ChemiDoc XRS+ Molecular Imager (Bio-Rad, Cat. #1708265). Blots were analyzed in Fiji.

Astrocyte Gene Expression.

For analysis of astrocyte gene expression, cells were washed after RAC treatment with PBS and processed for RNA extraction per manufacturer's protocol (Zymo Research, Cat. #R1050). Total RNA purity and concentration was assessed using NanoDrop (ThermoFisher). 500 ng of RNA was used for cDNA synthesis using High-Capacity cDNA Reverse Transcription Kit (Fisher Scientific, Cat. #43-749-66) and total volume of cDNA was diluted to a final concentration of 2 ng/μ1. Gene expression was assessed using a RT Profiler PCR Array (Qiagen, Cat. #330231) specific to mouse NF-κB Signaling Targets according to manufacturer's protocol.

Astrocyte Glutamate Uptake.

For analysis of astrocyte glutamate uptake, cells were washed after RAC treatment with PBS, then washed with Hank's balanced salt solution (HBSS). Fresh HBSS was added to each well and 100 μM glutamate (Sigma, Cat. #G1251) was added to each well. Empty wells not containing cells were filled with HBSS and 100 μM glutamate as a negative control. Cells were incubated at room temperature for 3 hours before HBSS was collected and glutamate concentration determined by colorimetric assay (Sigma, Cat. #MAK004). Relative change in glutamate uptake was calculated compared to negative control wells.

Neuronal Survival.

For analysis of neuronal survival, culture media was collected after RAC treatment and added directly to rat neurons (previously described above) for 24 hours. After exposure, neuronal viability was assessed with the ApoTox-Glo Triplex Assay according to manufacturer's protocol (Promega, Cat. #G6320).

### Results

Treatment with BGE-105 showed a decrease in inflammatory biomarkers in in vitro astrocyte such as RAC-induced astrocytes described below. The data demonstrated that administration of BGE-105 to reactive astrocyte cocktail (RAC)-stimulated astrocytes decreased proinflammatory reactive profile.

FIG. 8 illustrates results of gene profiling from Groups 1-4. BGE-105 attenuated RAC-induced CXCL1, CD3 and IL-6 gene expression. CXCL1, CD3 and IL-6 are typically driven by IL-1/TNF-mediated NF-κB activation. As shown in FIG. 8, expressions of biomarkers CXCL1, C3, and IL-6 were low in control (CON) astrocytes and BGE-105 (CON+105) which had no or minimal impact on their expression in astrocytes. CXCL1 is a chemokine that is typically upregulated in neurodegenerative disease, such as Alzheimer's disease. CXCL1 expression was significantly upregulated in RAC-induced reactive astrocytes (RAC) by about 1200-1600 fold (p<0.0001) when compared to the control (CON) group (left panel). Treatment with BGE-105 (RAC+105) significantly reduced CXCL1 expression when compared to the RAC group (p=0.0204).

C3 is a biomarker for reactive astrocyte. C3 expression was significantly upregulated in RAC-induced reactive astrocytes (RAC) by about 20-30 fold (p<0.0001) when compared to the control (CON) (middle panel). Treatment with BGE-105 (RAC+105) significantly reduced C3 expression by 10-20 fold (p=0.0015) as compared to the RAC group (RAC). The result indicated that BGE-105 was effective in reducing neurotoxicity in reactive astrocytes.

Reactive astrocytes in pathological conditions adversely affect endothelial integrity via secreted proteins, such as VEGF or IL-6. FIG. 8 right panel shows IL-6 was upregulated in RAC-induced reactive astrocytes (RAC) by about 10-15 fold (p<0.0001). Treatment with BGE-105 (RAC+105) significantly reduced IL-6 expression by about 5-10 fold (p<0.0001).

FIG. 9 shows measurements of biomarkers in the culture media obtained from Groups 1-4 and 6. BGE-105 (CON+105) had no or minimal impacts on concentration of CXCL1 and IL-6 detected in media cultured with astrocytes when compared to the control (CON). While FIG. 9 presents yields from Groups 1-4 and 6, it is noted that Group 6 has very low yield, which is too low to process alongside Groups 1-4.

FIG. 9 left panel shows treatment with BGE-105 (RAC+105, Group 4) greatly reduced astrocyte release of CXCL1, and thus the concentration of CXCL1 (p=0.0032). Reactive astrocytes (RAC, Group 3) increased release of CXCL1 by about 30000-40000 fold when compared to the control astrocytes (CON, Group 1) (p<0.0001). A similar result of RAC+BAY, Group 6 was observed for treatment with NF-κB inhibitor (RAC+BAY, p<0.0001).

FIG. 9 right panel shows reactive astrocytes (RAC) treated with BGE-105 (RAC+BGE-105, Group 4) reduced astrocytic release of IL-6 and thus reduced the concentration of IL-6 (p=0.0008). Reactive astrocytes (RAC) increased release of IL-6 by about 1600-2400 fold when compared to the control astrocytes (CON) (p<0.0001). A Similar result of Group 6 (RAC+BAY) was observed for treatment with NF-κB inhibitor (RAC+BAY, p<0.0001).

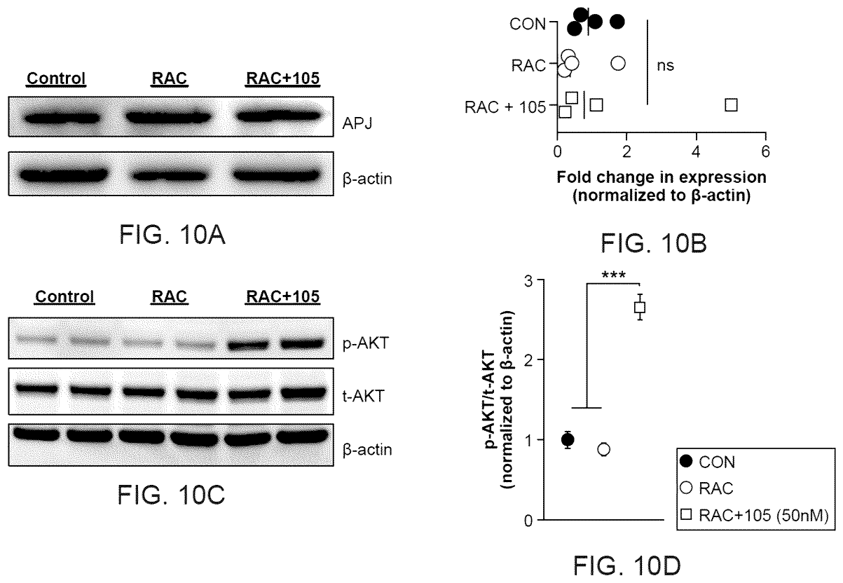

FIGS. 10A-10D show that BGE-105 activates apelin receptor (APJ) signaling in astrocytes. Shown are APJ, p-AKT, t-AKT and β-actin protein expressions in control astrocyte cells, cells treated with reactive astrocyte cocktail (RAC), and RAC cells treated with BGE-105 (RAC+BGE-105 [50 nM]). FIG. 10A shows that the expression of APJ was comparable in all three experimental groups (Control (CON), RAC, RAC+BGE-105). No significant fold change in expression was detected when normalized with β-actin expression (FIG. 10B). FIG. 10C shows expression of p-AKT was increased in the RAC+BGE-105 group, while expression of t-AKT remained comparable in all three experimental groups. Expression level of p-AKT was significantly increased in response to RAC+BGE-105 treatment by about 2 folds, as compared to t-AKT, and normalized with β-actin expression (FIG. 10D). p-AKT: phosphorylated AKT.

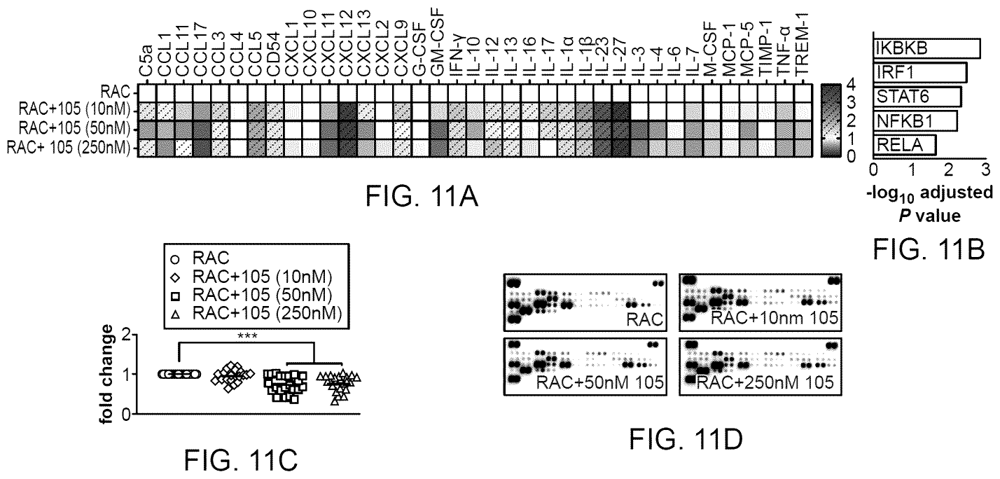

FIGS. 11A-11D show that BGE-105 decreases cytokine release in a dose-dependent manner. FIG. 11A shows relative fold change in expression of the various cytokines. The legend on the right ranges from 0 fold change (blue) to 1-fold change (or no change, labeled in white), up to 4+ fold change (dark red). FIG. 11A shows reduction of a panel of cytokines and chemokines release that was detected in astrocytes treated with reactive astrocyte cocktail (RAC) and various doses of BGE-105 (RAC+BGE-105 [10 nM, 50 nM, 250 nM]) in comparison to RAC. FIG. 11B shows transcription factor enrichment of biomarkers: IKBKB, IRF1, STATE, NF-κB1, and RELA FIG. 11C shows fold changes of cytokine and chemokine release detected from different treatments (RAC, RAC+105 (10 nM), RAC+105 (50 nM), RAC+105 (250 nM). FIG. 11C shows that there is a higher number of downregulated (or <1 fold change) cytokines as the dose of BGE-105 increases. Treatment with BGE-105 (RAC+BGE-105 [50 nM and 250 nM]) significantly reduced cytokine and chemokine release. FIG. 11D shows protein bands of cytokine and chemokine as quantified by western blot and presented in dot blots.

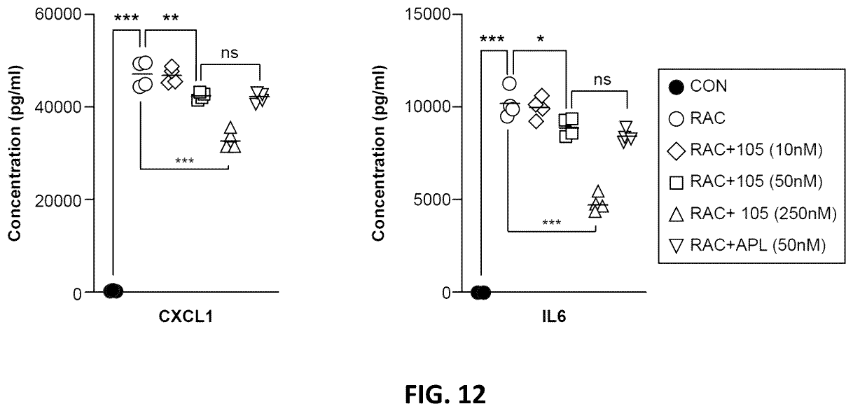

FIG. 12 shows treatment of reactive astrocytes with BGE-105 reduced astrocytic release of CXCL1 and IL-6. FIG. 12 left panel shows that astrocytes treated with reactive astrocyte cocktail (RAC) had increased concentration/release of CXCL1 by about 40000-60000 pg/ml as compared to the control (CON) group. BGE-105 (RAC+BGE-105 [50 nM, 250 nM]) significantly reduced CXCL1 concentration/release to about 40000 pg/ml (50 nM) or about 20000-40000 pg/ml (250 nM). It is noted that no significant difference of CXCL1 concentration/release was detected between the BGE-105 (RAC+BGE-105 [50 nM]) and apelin (APL) (RAC+APL [50 nM]) treatment groups. FIG. 12 right panel shows treatment with RAC increased concentration/release of IL-6 by about 10000 pg/ml as compared to the control (CON) group. BGE-105 (RAC+BGE-105 [50 nM, 250 nM]) significantly reduced IL-6 concentration/release to about 5000-10000 pg/ml (50 nM) or about 10000 pg/ml (250 nM). It is noted that no significant difference of IL-6 concentration/release was detected between the BGE-105 (RAC+BGE-105 [50 nM]) and apelin (APL) (RAC+APL [50 nM]) treatment groups.

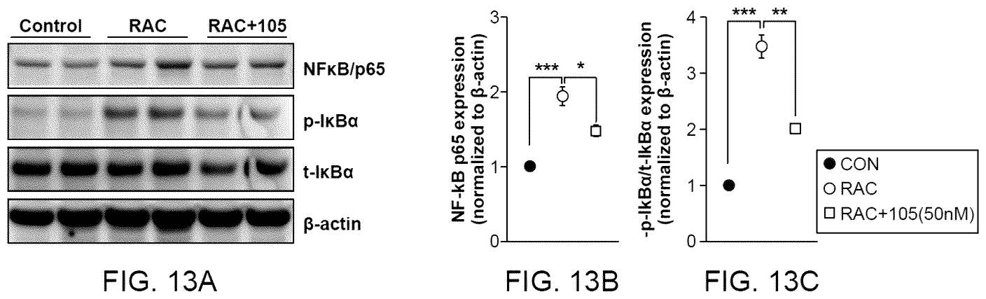

Nuclear factor-κB (NF-κB) consists of a family of transcription factors that play critical roles in inflammation, immunity, cell proliferation, differentiation, and survival. Inducible NF-κB activation depends on phosphorylation-induced proteosomal degradation of the inhibitor of NF-κB proteins (IκBs, such as κBα, κBβ), which retain inactive NF-κB dimers in the cytosol in unstimulated cells. Multiple studies have demonstrated that signaling through NF-κB in astrocytes contributes to pro-inflammatory responses following injury and that inhibition of NF-κB in astrocytes can promote functional recovery (Dresselhaus E C, Meffert M K, Cellular Specificity of NF-κB Function in the Nervous System. Front Immunol. 2019 May 9; 10:1043). FIGS. 13A-13C shows BGE-105 inhibited NF-κB activation and IκBα phosphorylation in astrocytes. FIG. 13A shows NF-κB, p-IκBα, t-IκBα, and β-actin protein expressions in control astrocyte cells, astrocytes treated with reactive astrocyte cocktail (RAC), and with BGE-105 (RAC+BGE-105 [50 nM]). FIG. 13B shows NF-κB p65 expression normalized to β-actin. RAC increased NF-κB p65 expression as compared to the control (CON).However, treatment with BGE-105 (RAC+BGE-105 (50 nM)) significantly reduced NF-κB p65 expression. FIG. 13C shows the ratio of p-IκBα/tIκBα expression normalized to β-actin. RAC had increased ratio of p-aBa/tIκBα expression and treatment with BGE-105 (RAC+BGE105) significantly reduced the ratio of p-IκBα/tIκBα expression.

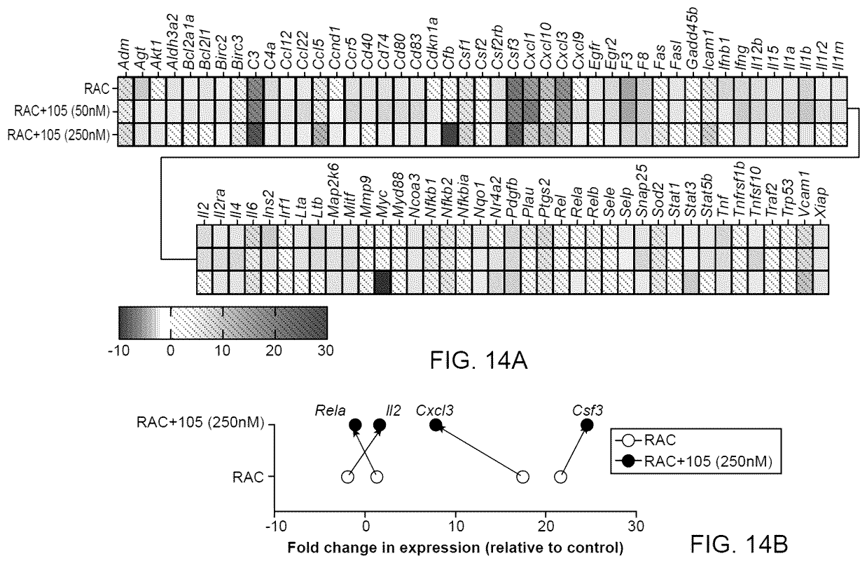

FIGS. 14A-14B show reactive astrocytes treated with BGE-105 had a dose-dependent effects on NF-κB signaling transcription response. FIG. 14A is a heat map illustrating expression of biomarkers associated with the NF-κB signaling pathway in astrocytes treated with reactive astrocyte cocktail (RAC), and in response to treatment of BGE-105 (RAC+BGE-105 [50 nM, 250 nM]). The legend shows fold change in expression compared to the control group (not shown), ranging from −10-fold change (blue) to 0-fold change (or no change, labeled in white), up to 30 fold change (dark red). FIG. 14B shows fold change in expression relative to control of exemplary subset of biomarkers from FIG. 14A: Rela, Il2, Cxcl3, and Csf3. Showing two cases of downregulation (Rela [decreased by 182%], Cxcl3 [decreased by 55%]) of proinflammatory genes and two cases of upregulation (Il2 increased by 184%], Csf3 [increased by 16%]) of anti-inflammatory/neurotrophic genes in response to treatment of reactive astrocytes with a higher dose of BGE-105 (250 nM) (RAC+BGE-105 [250 nM]).

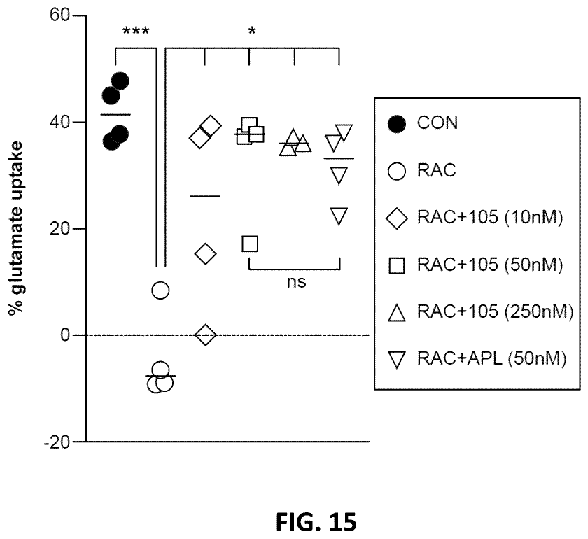

FIG. 15 shows that BGE-105 improved glutamate clearance deficit in RAC astrocytes. Glutamate clearance was significantly decreased in RAC astrocytes (RAC), which may contribute to glutamate excitotoxicity in neurodegeneration/aging. BGE-105 (RAC+BGE-105 [10 nM, 50 nM, 250 nM]) and apelin (APL) (RAC+APL [50 nM]) significantly and statistically increased the percentage of glutamate uptake.

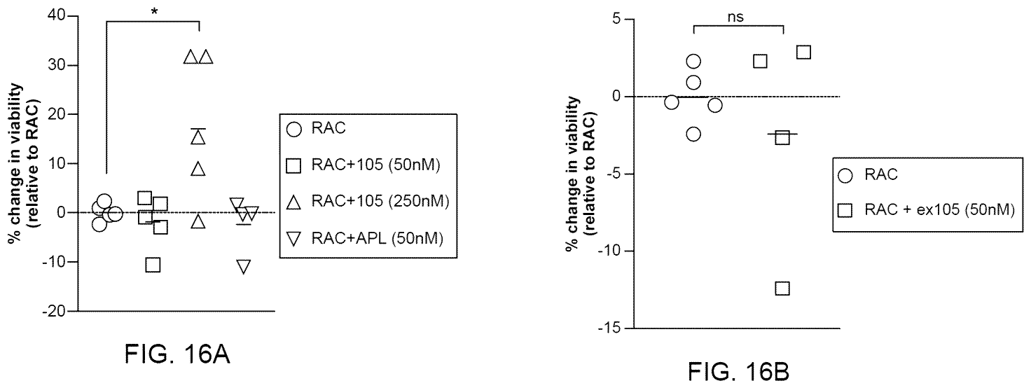

FIGS. 16A-16B show reactive astrocytes treated with BGE-105 improved cellular viability after RAC-conditioned media challenge. Shown is a percentage change in viability relative to RAC in astrocytes in control (RAC) and BGE-105 (RAC+BGE105 [50 nM, 250 nM]) and apelin (APL) (RAC+APL [50 nM]) treatment groups. FIG. 16A shows BGE-105 (RAC+BGE105 [250 nM]) significantly and statistically increased cellular viability by about 30% as compared to RAC.

To understand whether BGE-105 could directly act on neurons to be protective, or whether any protective mechanisms would require action on astrocytes, cells were treated with exogenous BEG-105 (i.e., BGE-105 was added directly to neurons in addition to the toxic RAC media). This approach is in opposition to the typical experimental conditions, where BGE-105 was added to the astrocytes when they were stimulated with the RAC components. As shown in FIG. 16B, treatment with exogenous BGE-105 (RAC+ex105 [50 nM]) did not provide statistical change of cell viability relative to RAC. The result shows that exogenous BGE-105 did not directly protect neurons against RAC-induced neurotoxicity.

In summary, these data demonstrated that the apelin receptor is abundantly expressed and activated by BGE-105 in astrocytes. The data demonstrated that BG5-105 dampens astrocytic inflammatory response following RAC exposure in mouse astrocyte cells. Additionally, the data show that BGE-105 limits RAC-induced astrocyte inflammation through modifications of NF-κB signaling. Overall, the data demonstrate that BGE-105 improved cellular function in astrocytes and protected against RAC-induced cell death in neurons. The results indicated BG5-105 is effective in reducing neurotoxicity in degenerative or aging astrocytes, and thus can be used for rescuing BBB permeability in a patient that has, or is suspected of having, a neurogenerative disease.

6.7. Example 7: Effects of BGE-105 on Neuroinflammation in Aged Mouse in a Traumatic Brain Injury (TBI) Model

This study assessed the effects of BGE-105 on mitigating the effects of CNS damage and inflammation, and accelerating functional recovery, in a mouse model of TBI.

BGE-105 is given orally to aged mice at various potential doses for a period of time before and/or after controlled cortical impact (model of TBI in mice); during the experimental period, mice undergo various behavioral assessments to understand whether BGE-105 treatment affected motor and cognitive functions known to be impaired by TBI. After, the brains of mice are analyzed for neuroinflammatory markers by a variety of histological and biochemical methods

### Result

Treatment with BGE-105 causes a decrease of neuroinflammatory markers and inhibit neuro-inflammation in aged mice with TBI.

6.8. Example 8: Effects of BGE-105 on Neural Stem Cells from Patient Derived Induced Pluripotent Stem Cells (iPSCs)

This study assesses the effect of BGE-105 (or media from other brain cells exposed to BGE-105, such as microglia and astrocytes) in human patient, iPSC-derived neurons improves a variety of functions associated with beneficial neuronal outcomes, including those listed below
i. proliferation and survival
ii. Autophagy
iii. synaptic plasticity
iv. ROS production

### Result

Treatment with BGE-105 improves overall survival, differentiation, and cellular functioning in human, stem-cell-derived neurons. Improvement is assessed by comparison to neural stem cells derived from iPSCs not treated with BGE-105 (control).

6.9. Example 9: Effects of BGE-105 on Astrocyte Apelinergic Signaling and Motor Neuron Function in a Mouse Model of ALS

The study accesses the effects of BGE-105 on astrocyte apelinergic signaling and motor neuron function. The study investigates anti-inflammatory and neurotrophic apelinergic signaling events in primary SOD1^{G93A} mouse astrocytes. The study characterizes the effects of BGE-105 on primary astrocytes derived from wildtype and SOD1^{G93A} mice in vitro. This includes measuring effects on gene expression, secretion of inflammatory cytokines, and glutamate uptake. Additionally, the study conducts co-culture experiments using SOD1^{G93A} astrocytes and wildtype motor neurons to determine effects of apelin signaling on rescue of neuronal survival and function. This is performed using measures of viability, neuronal homeostasis, and morphological endpoints.

The purpose of this study is to demonstrate the effects of BGE-105 on astrocytic apelinergic signaling and motor neuron function. Astrocyte reactivity plays a causal role in motor neuron loss, declining motor function, and shortened lifespan in preclinical mouse models of ALS (REF). Astrocyte dysfunction in ALS is driven by a microglia-derived proinflammatory signal comprised of IL-1α, TNFα, and C1q. In turn, this IL-1α/TNFα/C1q stimulus promotes proinflammatory NFκB-mediated signaling, leading to impaired astrocytic support and, ultimately, neuron death. BGE-105 is a potent agonist of the apelin receptor APJ, which is known to regulate NFκB activation (REF), mitigating inflammatory insults in vitro and in vivo. This study demonstrates, in primary cultures of astrocytes and neurons from the SOD1^{G93A} mouse model of ALS, that BGE-105 can alleviate astrocytic IL-1α/TNFα/C1q-mediated neurotoxicity and improve overall astrocytic and neuronal function. The data provide a mechanistic rationale for the effects of enhanced apelinergic signaling in the CNS toward application of BGE-105 treatment in well-validated mouse models of ALS.

6.10. Example 10: Investigate Anti-Inflammatory and Neurotrophic Apelinergic Signaling Events in Primary SOD1^{G93A} Mouse Astrocytes.

The study characterizes effects of BGE-105 on primary astrocytes derived from wildtype and SOD1^{G93A} mice in vitro. This includes measuring effects on gene expression, secretion of inflammatory cytokines, and glutamate uptake.

### Methods

The study utilizes primary mouse astrocyte and neuron cultures generated from the SOD1^{G93A} mouse model of familial ALS as previously described (Lundquist et al. 2019. Exercise induces region-specific remodeling of astrocyte morphology and reactive astrocyte gene expression patterns in male mice. Journal of Neuroscience Research, 97(9), 1081-1094) with some modifications. Briefly, whole spinal cords are resected from PNDO-4 (postnatal day) SOD1^{G93A+} pups and wildtype littermates. Spinal cords are enzymatically and mechanically processed before using MACS (magnetic activated cell sorting) to positively select for astrocytes with ACSA2-conjugated magnetic beads (Miltenyi Biotec) (REF). Astrocytes are cultured in serum-free conditions in T75 flasks for 7 days before splitting to 6- or 12-well plates for final experimentation.

Astrocytes from wildtype and SOD1^{G93A} mice are first studied in isolation to compare transcriptomic and cytokine release profiles. Next, vehicle or 250 nM BGE-105 (dissolved in DMSO in saline) is added to the wildtype or SOD astrocytes for 24 hours. Then, either media is collected, and cells are lysed for transcriptomic and protein analysis, or cells are incubated with 100 μM glutamate in HBSS (Hanks' Balanced Salt Solution) for three hours to monitor glutamate uptake using commercially available, colorimetric kits (Sigma). ACM (astrocyte-conditioned media) are analyzed for release of cytokines and neurotrophic factors using Mouse Proteome Profiler Arrays (R&D Systems), and commercially available ELISAs (for select cytokines from protein arrays). Astrocytic gene expression are analyzed by RT-qPCR, and astrocyte protein expression are analyzed by western blotting as previously described (Lundquist et al, 2022).

### Results

SOD1^{G93A} astrocytes treated with BGE-105 release less proinflammatory cytokines into the astrocyte conditioned media (ACM). These cytokines are regulated by NF-κB stabilization, and genetic analysis of untreated and BGE-105 treated SOD1^{G93A} astrocytes reveal changes to NF-κB signaling activity, resulting in less cytokine production.

6.11. Example 11: Effects of Apelin Signaling on Rescue of Neuronal Survival and Function.

This study conducts co-culture experiments using SOD1^{G93A} astrocytes and wildtype motor neurons to determine effects of apelin signaling on rescue of neuronal survival and function. Co-culture experiments are conducted by measuring viability, neuronal homeostasis, and morphological endpoints.

### 7. METHODS

Neurons are isolated from mouse spinal cords as previously described in Example 12 above. Non-astrocytic flowthrough following column-based cell isolation (MACS) are collected and cultured in serum-free, neuron-selective media in 12- or 96-well plates for at least 14 days before experimentation. For specific selection of spinal cord motor neurons, wildtype C₅₇BL/6J mouse PNDO or adult spinal cords are processed using published methods (Beaudet, 2015).

Neurons are cultured for at least 14 days before the start of experimentation. First, ACM from wildtype or SOD astrocytes exposed to 250 nM BGE-105 (or vehicle) are added to neurons seeded in 96 well plates for 6, 12, 24, or 48 hours followed by analysis of neuronal survival using the ApoTox-Glo Triplex kit (Promega) to determine optimal exposure time. Next, ACM from wildtype or SOD astrocytes exposed to 250 nM BGE-105 (or vehicle) are added to neurons seeded in 12-well plates before collecting neuronal protein lysates for analysis of synaptic protein markers synaptophysin and PSD-95 by western blotting as previously described (Lundquist et al., 2022).

Next, SOD1^{G93A} or wildtype astrocytes are added into neurons seeded in 96-well plates and grown together for the next 10 days. Independent astrocyte-neuron cocultures are continuously treated with 250 nM BGE-105, fixed with 4% PFA (paraformaldehyde) at one, three, five, and 10 days, stained with fluorescent antibodies against synaptic and axonal markers (synaptic—synaptophysin, PSD95, Bassoon, Homer; axonal—MAP2, β-III tubulin), and imaged using a high-content imager (CX5, Thermofisher) to assess morphological endpoints.

The overall male/female ratio of newborn litters of mouse pups cannot be controlled. To account for possible effects of sex as a biological variable, the sex of all mice is tracked from each litter, ensuring equal numbers of male and female pups of either genotype are utilized in experiments across litters. All primary mouse cell culture experiments are repeated using at least three independent preparations from several litters of mice.

### Results

Treatment of neurons with SOD1^{G93A} ACM results in higher cell death, which is attenuated by pretreatment of SOD1^{G93A} astrocytes with BGE-105. Additionally, BGE-105 preserves synaptic protein expression and neuronal structural integrity when wildtype neurons are cultured with SOD1^{G93A} astrocytes.

7.1. Example 12: Effects of BGE-105 in a Mouse Model of ALS

The study accesses the effects of BGE-105 in a mouse model of ALS. The study establishes pharmacokinetic/pharmacodynamic features of intracerebroventricular (ICV) administration of BGE-105 in wildtype C57BL/6J mice. Multiple doses across multiple timepoints are tested to determine drug characteristics within the central nervous system (CNS). BGE-105 is administered to SOD1^{G93A} mice using Alzet minipumps connected to ICV cannulas.

Astrocyte reactivity, driven by IL-la/TNFα/C1q-mediated neurotoxicity, contributes to spinal cord motor neuron death and motor dysfunction in mouse models of ALS. Triple transgenic knockout of IL-1α/TNFα/C1q in the SOD1^{G93A} mouse model of ALS dramatically extends lifespan and motor neuron survival (Guttenplan, 2020). Preliminary in vitro evidence as shown in Example 6 suggests that IL-1α/TNFα/C1q-mediated astrocyte reactivity, highlighted by proinflammatory cytokine release and impaired glutamate recycling, is dampened by administration of BGE-105. This study first validates intraventricular administration of BGE-105 into wildtype mice to establish CNS-specific drug properties and guide optimal dose selection. Next, the efficacy of CNS delivery of BGE-105 to minimize motor neuron death, improve motor function, and extend lifespan in the SOD1^{G93A} mouse model of ALS is tested. For experiments in vivo, BGE-105 are administered to SOD1^{G93A} mice using Alzet minipumps. Alzet minipumps are implanted at 4 months of age, followed by 1 month of continuous administration of BGE-105.

Analyses of motoric, behavioral, and survival endpoints are measured. Brain, spinal cord, and skeletal muscle are dissected post mortem for histochemical and transcriptomic analyses to determine target engagement and mechanisms of action.

Result:

Treatment of mice with BGE-105 minimizes motor neuron death, improves motor function, and extends lifespan in the SOD1^{G93A} mouse model of ALS

Establishment of Pharmacokinetic and Pharmacodynamic Properties of Intracerebroventricular (ICV) Administration of BGE-105 in Wildtype Mice.

BGE-105 is administered into the lateral ventricles of 4-month-old C57BL/6J mice via stereotactic surgery at 1, 5 or 25 mg BGE-105/kg bodyweight, followed by tissue collection at 1, 8, and 24 hours (n=27 mice total [3 mice per dose, per time point]). Alternative delivery routes such as direct CNS delivery of BGE-105 are used to achieve sufficient exposure throughout the spinal cord. Blood and CSF (cerebrospinal fluid) are drawn by retroorbital bleed and cisterna magna puncture, respectively, before mice are perfused with ice-cold normal saline and whole brains and spinal cords are resected, divided into two halves, and flash frozen in liquid nitrogen. Whole blood is spun to separate and collect the plasma; plasma, CSF, and one half of each tissue sample for every mouse is sent for drug exposure measurements by LC-MS (liquid chromatography-mass spectrometry) at Quintara Discovery, Inc (Hayward, CA). The other half of brain and spinal cord are processed for protein analysis by mechanical digestion in N-PER Extraction Reagent (Thermofisher) with Halt Protease/Phosphatase Inhibitor (Thermofisher) before analysis by western blotting for phosphorylated AKT, a marker of Gᵢ-PCR activity, to determine APJ engagement throughout the CNS.

Investigation of Efficacy of ICV Administration of BGE-105 in SOD1^{G93A} Mouse Model of ALS.

Alzet minipumps connected to intraventricular cannulas are utilized for continuous CNS delivery of BGE-105, akin to previous studies on APJ agonism in the CNS (Zhu et al., 2020. Apelin-36 mediates neuroprotective effects by regulating oxidative stress, autophagy and apoptosis in MPTP-induced Parkinson's disease model mice, Brain Research, Volume 1726, 2020, 146493, ISSN 0006-8993). 4-month-old SOD1^{G93A} mice (C57BL/6J background; strain #004435, Jackson Laboratory) of both sexes (n=12/group; 6 male, 6 female) are randomized into vehicle or two separate treatment groups before undergoing baseline motor behavior assessments including the open field, grid hang, beam walk, and accelerating rotarod. Next, bilateral cannulas are targeted to the lateral ventricles using mouse stereotactic coordinates (Paxinos and Franklin, 2019) and fixed to the skull with dental cement. Alzet minipumps (model #2004) filled with BGE-105 at two doses (low and high, based upon results of the PK/PD study described above, or vehicle is implanted subcutaneously along the back and connected via catheter to the cannulas.

After recovery from surgery, mice are single housed for 28 days in cages with running wheels connected to wireless, continuous data monitoring. After 28 days of BGE-105 dosing, mice repeat motor behavior assessments (open field, grip strength, beam walk, and accelerating rotarod) before being euthanized for molecular and histological assessments.

For molecular endpoints (n=6/group; 3 male, 3 female), mice are anesthetized with isoflurane, perfused with ice-cold normal saline, and cervically dislocated before whole brains and spinal cords are independently resected, weighed, and flash frozen in liquid nitrogen. Samples are processed for protein analysis by mechanical digestion in N-PER Extraction Reagent (Thermofisher) with Halt Protease/Phosphatase Inhibitor (Thermofisher) before analysis by western blotting. Markers of APJ activation (phosphorylated AKT), synaptogenesis (synaptophysin, PSD95), and neuroinflammation (IBA1, GFAP) are assessed in the brain and spinal cord.

For histological assessments (n=6/group; 3 male, 3 female), mice are anesthetized with isoflurane, and perfused with ice-cold normal saline, followed by ice-cold 4% PFA. Whole brains and spinal cords are independently resected and transferred to 4% PFA for overnight post-fixture at 4° C., followed by incubation in 30% sucrose at 4° C. until tissue sinks. Separately, the gastrocnemius is dissected and transferred to ice-cold 1% PFA overnight at 4° C., followed by incubation in 30% sucrose until tissue sinks.

Tissues are flash frozen in chilled isopentane and stored at −80° C. before embedding in OCT and sectioning on a cryostat for histological analysis. Whole brains are sectioned at 30-micron thickness, transferred onto gelatin-coated slides, and stored at −80° C. until further use. Spinal cords and gastrocnemius are sectioned at 14-micron thickness, transferred onto gelatin-coated slides, and stored at −80° C. until further use.

Brain sections are used for measurements of astrocytic and microglial reactivity (GFAP and IBA1, respectively) as previously described (Lundquist et al., 2019), as well as motor cortex cell density via Nissl staining, as previously described (Ozdinler et al., 2011. Corticospinal motor neurons and related subcerebral projection neurons undergo early and specific neurodegeneration in hSOD1G⁹³ A transgenic ALS mice. J Neurosci. 2011 Mar. 16; 31(11):4166-77; Lundquist et al., 2022), imaged and relative glial cell reactivity and cortical thickness are measured by a counter blinded to treatment conditions.

Spinal cords are stained for motor neuron density via Nissl staining, as previously (Lundquist, et al., 2022). Briefly, frozen sections are rehydrated, submerged in 0.1% cresyl violet solution for 5 minutes, and immediately washed in distilled water before undergoing dehydration in 100% ethanol and clearing in xylene. Slides are cover slipped, sealed, imaged, and analyzed by a counter blinded to condition to assess motor neuron density.

Gastrocnemius sections are processed for neuromuscular junction innervation using fluorescent immunohistochemistry as previously described (Guttenplan et al. 2020. Knockout of reactive astrocyte activating factors slows disease progression in an ALS mouse model. Nat Commun 11, 3753) with modifications. Briefly, sections are permeablized with 0.1% Trition in TBS (Tris buffered saline) for 1 hour at room temperature, followed by blocking in 10% normal goat serum for 2 hours at room temperature and overnight incubation with neurofilament heavy chain (NF—H) antibody (rabbit anti-NF—H, Abcam) at 4° C. Sections are washed in TBS before incubation with a goat anti-rabbit fluorescent secondary in addition to Alexa 488-conjugated a-bungarotoxin (Invitrogen) for 2 hours at room temperature. Sections are washed in TBS, coverslipped with VECTASHIELD nuclear counter stain mounting medium (Vector), imaged, and analyzed by a counter blinded to condition to assess neuromuscular junction innervation.

Mice undergo daily health checks and be weighed three times per week to assess animal welfare and survival, and catheters are checked daily for patency.

### Results

The data demonstrates the feasibility and technical merit of developing the drug BGE-105 (and related compounds) as a safe and efficacious treatment to slow progression and reduce severity of amyotrophic lateral sclerosis (ALS).

7.2. Example 13: Effects of BGE-105 on Immune Biomarkers in Humans

The study described herein characterizes the effects of BGE-105 on immune biomarkers in human in a Phase 1b clinical trial directed to treatment of muscular atrophy. BGE-105 is a highly selective, potent, orally available small-molecule agonist of the apelin receptor APJ. BGE-105 treatment resulted in statistically significant prevention of muscle atrophy relative to placebo in healthy volunteers aged 65 or older after 10 days of strict bed rest. The data show change of gene expression of immune biomarkers in response to BGE-105 treatment.

### Study Design and Results

The double-blind, placebo-controlled trial evaluated the safety and pharmacodynamics of BGE-105. Twenty-one volunteers underwent 10 days of bed rest while receiving infusions of BGE-105 or placebo.

Biological samples from treated and placebo groups were collected on Day 6 and Day 11. Fold change of immune biomarker expression was measured and summarized in Table 2.

TABLE 2
Effect of BGE-105 on immune biomarkers in human

| Entrez Gene Symbol | Target Full Name | Target |
|---|---|---|
| CCL2 | C-C motif chemokine 2 | MCP-1 |
| IL10 | Interleukin-10 | IL-10 |
| CCL11 | Eotaxin | Eotaxin |
| CCL5 | C-C motif chemokine 5 | RANTES |
| CXCL1 | Growth-regulated alpha protein | Gro-a |
| Il16 | Interleukin-16 | Il-16 |
| CXCL11 | C-X-C motif chemokine 11 | I-TAC |

The drug was well tolerated in the study. This human data supports the use of BGE-105 to reduce inflammation as a mechanism for restoring the BBB, and extends to treatment, prevention or reduction of a disorder or condition associated with BBB permeability, such as neurodegenerative diseases or related conditions, including Alzheimer's disease (AD), vascular dementia (VaD), delirium, cognitive impairment, Parkinson's disease (PD), amyotrophic lateral sclerosis (ALS), stroke, Huntington's disease (HD), multiple sclerosis (MS), and traumatic brain injury (TBI).

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of treating a disease or disorder associated with blood-brain barrier (BBB) permeability in a subject in need thereof, the method comprising administering to the subject a therapeutically effective amount of an apelin receptor.

2. The method of claim 1, wherein the condition or disorder is associated with increased BBB permeability.

3. The method of claim 1, wherein the disease is a neurodegenerative disease.

4. The method of claim 3, wherein the neurodegenerative disease is selected from Alzheimer's disease (AD), Parkinson's disease (PD), amyotrophic lateral sclerosis (ALS), stroke, Huntington's disease (HD), multiple sclerosis (MS).

5. The method of claim 1, wherein the disorder is cognitive impairment.

6. The method of claim 1, wherein the disorder is delirium.

7. The method of claim 6, wherein the subject has intensive care unit (ICU) delirium, post-operative delirium, delirium due to trauma, or delirium due to trauma from a hip fracture or cardiovascular surgery.

8. The method of claim 1, wherein the disorder is chronic or progressive dementia.

9. The method of claim 1, wherein the disorder is traumatic brain injury (TBI).

10. The method of claim 1, wherein the subject has acute cognitive impairment.

11. The method of claim 10, wherein the subject has postoperative cognitive dysfunction (POCD).

12. The method of claim 1, wherein the subject is on a ventilator.

13. The method of claim 1, wherein the condition is neurodegeneration.

14. The method of claim 1, wherein the subject has neuroinflammation.

15. The method of claim 1, wherein the subject is human and at least 40-years-old.

16. The method of claim 15, wherein the subject is at least 50-years-old.

17. The method of claim 16, wherein the subject is at least 60-years-old.

18. The method of claim 17, wherein the subject is at least 65-years-old.

19. The method of claim 18, wherein the subject is at least 70-years-old.

20. The method of claim 19, wherein the subject is at least 75-years-old.

21. The method of claim 20, wherein the subject is at least 80-years-old.

22. The method of claim 1, wherein the subject has, or is identified as having, a low circulating level of apelin.

23. The method of claim 1, wherein the apelin receptor agonist is of formula (I) or (II): 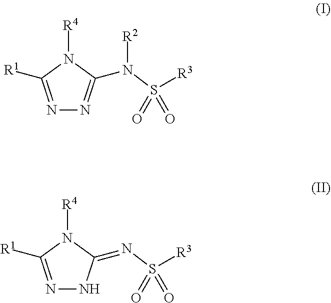
or a pharmaceutically acceptable salt thereof, a tautomer thereof, a pharmaceutically acceptable salt of the tautomer, a stereoisomer of any of the foregoing, or a mixture thereof,
wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3b} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula (CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

24-37. (canceled)

38. The method of claim 23, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide or a pharmaceutically acceptable salt thereof.

39. The method of claim 1, wherein the apelin receptor agonist is administered intravenously or intrathecally.

40. The method of claim 1, wherein the apelin receptor agonist is administered orally.

41-47. (canceled)

48. The method of claim 1, wherein the apelin receptor agonist is administered daily.

49. The method of claim 1, wherein the apelin receptor agonist is administered as a plurality of equally or unequally divided sub-doses.

50. The method of claim 1, wherein the apelin receptor agonist is administered at varying dosing intervals.

51. The method of claim 1, wherein the apelin receptor agonist is administered at a dose of 200 mg.

52. The method of claim 1, further comprising, assessing cognitive function after the dosing.

53. The method of claim 52, wherein the cognitive function is assessed at least one day after dosing.
